(19) United States
(12) Patent Application Publication
(10) Pub. No.: US 2006/0128721 A1
(43) Pub. Date: Jun. 15, 2006

(54) Novel formulations of opioid-based treatments of pain comprising substituted 1,4-di-piperidin-4-yl-piperazine derivatives

(76) Inventors: Frans Eduard Janssens, Beerse (BE); Francois Maria Sommen, Beerse (BE); Benoit Christian Albert Ghislain De Boeck, Beerse (BE); Joseph Elisabeth Leenaerts, Beerse (BE); Yves Emiel, Maria Van Roosbroeck, Beerse (BE); Theo Frans Meert, Beerse (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/560,476

(22) PCT Filed: Jun. 7, 2004
(86) PCT No.: PCT/EP04/51048

§ 371 Date: Dec. 12, 2005

(30) Foreign Application Priority Data
Jun. 10, 2003 (WO) PCT/EP03/50220

(51) Int. Cl. A61K 31/496

(52) U.S. Cl. 514/253.12

(57) ABSTRACT

This invention concerns novel formulations for opioid-based treatments of pain and/or nociception comprising opioid analgesics and 1,4-di-piperidin-4-yl-piperazine derivatives having neurokinin antagonistic activity, in particular NK₁ antagonistic activity, the use of said formulation for the manufacture of a medicament for the prevention and/or treatment of emesis, pain and/or nociception, in particular in opioid-based acute and chronic pain treatments, more in particular in inflammatory, post-operative, emergency room (ER), breakthrough, neuropathic and cancer pain treatments and the use of an NK₁-receptor antagonist for the manufacture of a medicament for the prevention and/or treatment of respiratory depression in opioid-based treatments of pain.

The pharmaceutical formulations according to the invention comprise a pharmaceutically acceptable carrier and, as active ingredients, a therapeutically effective amount of an opioid analgesic and NK₁-antagonists according to the general Formula (I) 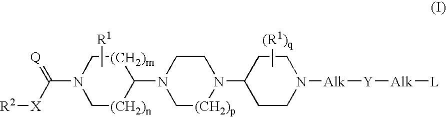 the pharmaceutically acceptable acid or base addition salts thereof, the stereochemically isomeric forms thereof, the N-oxide form thereof and prodrugs thereof, wherein all substituents are defined as in claim 1. The pharmaceutical composition according to the invention reduces to a large extent a number of unwanted side-effects associated with opioid analgesics, in particular emesis, respiratory depression and tolerance, thereby increasing the total tolerability of said opioids in pain treatment.

### FIELD OF THE INVENTION

This invention concerns novel formulations for opioid-based treatments of pain and/or nociception comprising opioid analgesics and 1,4-di-piperidin-4yl-piperazine derivatives having neurokinin antagonistic activity, in particular NK₁ antagonistic activity, the use of said formulation for the manufacture of a medicament for the prevention and/or treatment of emesis, in particular nausea and vomiting, pain and/or nociception, in particular in opioid-based acute and chronic pain treatments, more in particular in inflammatory, post-operative, emergency room (ER), breakthrough, neuropathic and cancer pain treatments and the use of an NK₁-receptor antagonist for the manufacture of a medicament for the prevention and/or treatment of emesis, in particular nausea and vomiting, respiratory depression and tolerance in opioid-based treatments of pain.

### BACKGROUND OF THE INVENTION

Opioid analgesics are the cornerstone of pain treatment, especially in the segment of moderate to severe acute and chronic pain. However, side-effects such as nausea/vomiting, constipation, respiratory depression and tolerance limit their use. The lowering of the high incidence of nausea and vomiting with many clinically used opioids is particularly considered as a major unmet medical need.

Neurokinins belong to a family of short peptides that are widely distributed in the mammalian central and peripheral nervous system (Bertrand and Geppetti, Trends Pharmacol. Sci. 17:255-259 (1996); Lundberg, Can. J. Physiol. Pharmacol. 73:908-914 (1995); Maggi, Gen. Pharmacol 26:911-944 (1995); Regoli et al., Pharmacol. Rev. 46 (1994)). They share the common C-terminal sequence Phe-Xaa-Gly-Leu-Met-NH₂. Neurokinins released from peripheral sensory nerve endings are believed to be involved in neurogenic inflammation. In the spinal cord/central nervous system, neurokinins may play a role in pain transmission/perception and in some autonomic reflexes and behaviors. The three major neurokinins are Substance P (SP), Neurokinin A (NK_{A}) and Neurokinin B (NK_{B}) with preferential affinity for three distinct receptor subtypes, termed NK₁, NK₂, and NK₃, respectively. However, functional studies on cloned receptors suggest strong functional cross-interaction between the 3 neurokinins and their corresponding receptors (Maggi and Schwartz, Trends Pharmacol Sci. 18: 351-355 (1997)). Species differences in structure of NK₁ receptors are responsible for species-related potency differences of NK₁ antagonists (Maggi, Gen. Pharmacol. 26:911-944 (1995); Regoli et al., Pharmacol. Rev. 46(4):551-599 (1994)). The human NK₁ receptor closely resembles the NK₁ receptor of guinea-pigs and gerbils but differs markedly from the NK₁ receptor of rodents. The development of neurokinin antagonists has led to date to a series of peptide compounds of which might be anticipated that they are metabolically too labile to be employed as pharmaceutically active substances (Longmore J. et al., DN&P 8(1):5-23 (1995)). NK₁-antagonists have been studied for a wide variety of indications including emesis, (stress-related) anxiety states, inflammatory responses, smooth muscle contraction and pain perception. NK₁-antagonists are in development for indications such as emesis, anxiety and depression, irritable bowel syndrome (IBS), circadian rhythm disturbances, visceral pain, neurogenic inflammation, asthma, micturition disorders, pancreatitis and nociception.

It has now surprisingly been found that a particular class of compounds with predominantly NK₁-activity reduces to a large extent a number of unwanted side-effects associated with opioid analgesics, thereby increasing the total tolerability of said opioids in pain treatment, in particular in opioid-based acute and chronic pain treatments, more in particular in inflammatory, post-operative, emergency room (ER), breakthrough, neuropathic and cancer pain treatments. More specifically, it was found in opioid-based treatments of pain that emesis was inhibited, respiratory depression was reduced, the tolerance for opioids was prevented and constipation was not worsened. Also, due to the intrinsic antinociceptive activity of NK₁-antagonists, even some increase in opioid efficacy is noted, thereby creating the option to reduce the opioid dose without effecting its analgesic action. Finally, by this combination, psychotropic properties were added to the analgesic efficacy by reducing stress, anxiety and depression.

### BACKGROUND PRIOR ART

Neurokinin antagonists are well known in the art (see for an overview e.g. U.S. Pat. No. 5,880,132) and exhibit a variety of non-related chemical structures.

Compounds containing the 1-piperidin-4-yl-piperazinyl moiety were disclosed in WO 97/16440-A1, published May 9, 1997 by Janssen Pharmaceutica N.V. for use as substance P antagonists, in WO 02/32867, published Apr. 25, 2002 by Glaxo Group Ltd. for their special advantages as neurokinin antagonists (more specifically were disclosed 4-piperazin-1-yl-piperidine-1-carboxylic acid amide derivatives), in WO 01/30348-A1, published May 03, 2001 by Janssen Pharmaceutica N.V., for use as substance P antagonists for influencing the circadian timing system, and in WO 02/062784-A1, published Aug. 15, 2002 by Hoffmann-La Roche AG for use as NK₁ antagonists.

Formulations containing NK₁-antagonists and opioid analgesics for the prevention and/or treatment of pain and/or nociception are disclosed in WO 96/20009 (Merck, Jul. 4, 1996), U.S. Pat. No. 5,880,132 (Merck, Mar. 9, 1999) and WO 97/25988 (Eli Lilly, Jul. 24, 1997). There is no mentioning of the reduction of side-effects apart from emesis.

The compounds of the present invention differ from the compounds of the prior art in the substitution of the piperazinyl moiety, being a substituted piperidinyl moiety as well as in their improved ability as potent, orally and centrally active neurokinin antagonists with therapeutic value in combinations with opioid analgesics for reduction of certain opioid-induced side-effects and increasing the tolerability of said opioids.

The compounds per se are disclosed in our co-pending application WO 2004/033428 A1 (Janssen Pharmaceutica, Apr. 22, 2004) as well as their use as neurokinin antagonists.

### DESCRIPTION OF THE INVENTION

The present invention relates to a pharmaceutical composition comprising a pharmaceutically acceptable carrier and, as active ingredients, a therapeutically effective amount of an opioid analgesic and a compound according to Formula (I) 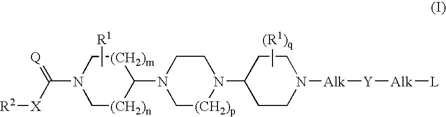
the pharmaceutically acceptable acid or base addition salts thereof, the stereochemically isomeric forms thereof, the N-oxide form thereof and prodrugs thereof, wherein:
n is an integer, equal to 0, 1 or 2;
m is an integer, equal to 1 or 2, provided that if m is 2, then n is 1;
p is an integer equal to or 2;
Q is O or NR³;
X is a covalent bond or a bivalent radical of formula —O—, —S— or —NR³—;
each R³ independently from each other, is hydrogen or alkyl;
each R¹ independently from each other, is selected from the group of Ar¹, Ar¹-alkyl and di(Ar¹)-alkyl;
q is an integer equal to 0 or 1;
R² is alkyl, Ar², Ar²-alkyl, Het¹ or Het¹-alkyl;
Y is a covalent bond or a bivalent radical of formula —C(=O)— or —SO₂—;
each Alk represents, independently from each other, a covalent bond; a bivalent straight or branched, saturated or unsaturated hydrocarbon radical having from 1 to 6 carbon atoms; or a cyclic saturated or unsaturated hydrocarbon radical having from 3 to 6 carbon atoms; each radical optionally substituted on one or more carbon atoms with one or more alkyl, phenyl, halo, cyano, hydroxy, formyl and amino radicals;
L is selected from the group of hydrogen, alkyloxy, Ar³-oxy, alkyloxycarbonyl, mono- and di(alkyl)amino, mono-and di(Ar³)amino, Ar³, is Ar³-carbonyl, Het² and Het²-carbonyl;
Ar¹ is phenyl, optionally substituted with 1, 2 or 3 substituents each independently from each other selected from the group of halo, alkyl, cyano, aminocarbonyl and alkyloxy;
Ar² is naphthalenyl or phenyl, each optionally substituted with 1, 2 or 3 substituents, each independently from each other, selected from the group of halo, nitro, amino, mono- and di(alkyl)amino, cyano, alkyl, hydroxy, alkyloxy, carboxyl, alkyloxycarbonyl, aminocarbonyl and mono- and di(alkyl)aminocarbonyl;
Ar³ is naphthalenyl or phenyl, optionally substituted with 1, 2 or 3 substituents each independently from each other selected from the group of alkyloxy, alkyl, halo, hydroxy, pyridinyl, morpholinyl, pyrrolidinyl, imidazo[1,2-a]pyridinyl, morpholinylcarbonyl, pyrrolidinylcarbonyl, amino and cyano;
Het¹ is a monocyclic heterocyclic radical selected from the group of pyrrolyl, pyrazolyl, imidazolyl, furanyl, thienyl, oxazolyl, isoxazolyl, thiazolyl, isothiazolyl, pyridinyl, pyrimidinyl, pymzinyl and pyridazinyl; or a bicyclic heterocyclic radical selected from the group of quinolinyl, quinoxalinyl, indolyl, benzimidazolyl, benzoxaazolyl, benzisoxazolyl, benzothiazoyl, benzisothiazolyl, benzofuranyl and benzothienyl; each monocyclic and bicyclic heterocyclic radical may optionally be substituted on any atom by a radical selected from the group of halo and alkyl;
Het² is a monocyclic heterocyclic radical selected from the group of pyrrolidinyl, dioxolyl, imidazolidinyl, pyrrazolidinyl, piperidinyl, morpholinyl, dithianyl, thiomorpholinyl, piperazinyl, imidazolidinyl, tetrahydroturanyl, 2H-pyrrolyl, pyrrolinyl, imidazolinyl, pyrrazolinyl, pyrrolyl, imidazolyl, pyrazolyl, triazolyl, furanyl, thienyl, oxazolyl, isoxazolyl, thiazolyl, thiadiazolyl, isothiazolyl, pyridinyl, pyrimidinyl, pyrazinyl, pyridazinyl and triazinyl; or a bicyclic heterocyclic radical selected from the group of benzopiperidinyl, quinolinyl, quinoxalinyl, indolyl, isoindolyl, chromenyl, benzimidazolyl, imidazo[1,2-a]pyridinyl, benzoxazolyl, benzisoxazolyl, benzothiazolyl, benzisothiazolyl, benzofuranyl and benzothienyl; each monocyclic and bicyclic radical optionally substituted with one or more radicals selected from the group of Ar¹, Ar¹alkyl, halo, hydroxy, alkyl, piperidinyl, pyrrolyl, thienyl, oxo, alkyloxy, alkyloxyalkyl and alkyloxycarbonyl; and
alkyl is a straight or branched saturated hydrocarbon radical having from 1 to 6 carbon atoms or a cyclic saturated hydrocarbon radical having from 3 to 6 carbon atoms; optionally substituted on one or more carbon atoms with one or more radicals selected from the group of phenyl, halo, cyano, oxo, hydroxy, formyl and amino radicals.

More in particular, the invention relates to a pharmaceutical composition comprising a pharmaceutically acceptable carrier and, as active ingredients, an opioid analgesic and a therapeutically effective amount of a compound according to Formula (I), the pharmaceutically acceptable acid or base addition salts thereof, the stereochemically isomeric forms thereof, the N-oxide form thereof and a prodrug thereof, wherein:
n is 1;
m is 1;
p is 1;
Q is O;
X is a covalent bond;
each R¹ is Ar¹ or Ar¹-alkyl;
q is 0 or 1;
R² is a Ar²;
Y is a covalent bond or a bivalent radical of formula —C(=O)— or —SO₂—;
each Alk represents, independently from each other, a covalent bond; a bivalent straight or branched, saturated or unsaturated hydrocarbon radical having from 1 to 6 carbon atoms; or a cyclic saturated or unsaturated hydrocarbon radical having from 3 to 6 carbon atoms; each radical optionally substituted on one or more carbon atoms with one or more phenyl, halo, cyano, hydroxy, formyl and amino radicals;
L is selected from the group of hydrogen, alkyloxy, Ar³-oxy, alkyloxycarbonyl, mono- and di(alkyl)amino, mono-and di(Ar³)amino, Ar³ and Het²;
Ar¹ is phenyl, optionally substituted with 1, 2 or 3 alkyl radicals;
Ar² is phenyl, optionally substituted with 1, 2 or 3 alkyl radicals;
Ar³ is phenyl, optionally substituted with 1, 2 or 3 substituents each independently from each other selected from the group of alkyloxy, alkyl, halo, hydroxy, pyridinyl, morpholinyl, pyrrolidinyl, imidao[1,2-a]pyridinyl, morpholinylcarbonyl, pyrrolidinylcarbonyl, amino and cyano;
Het² is a monocyclic heterocyclic radical selected from the group of pyrrolidinyl, piperidinyl, morpholinyl, pyrrolyl, imidazolyl, pyrazolyl, furanyl, thienyl, isoxazolyl, thiazolyl, thiadiazolyl, pyridinyl, pyrimidinyl, pyrazinyl, and pyridazinyl; or a bicyclic heterocyclic radical selected from the group of benzopiperidinyl, quinolinyl, quinoxalinyl, indolyl, chromenyl and benzimidazolyl; each monocyclic and bicyclic radical optionally substituted with one or more radicals selected from the group of Ar¹, Ar¹alkyl, halo, hydroxy, alkyl, piperidinyl, pyrrolyl, thienyl, oxo and alkyloxycarbonyl; and alkyl is a straight hydrocarbon radical having 1 to 6 carbon atoms, optionally substituted with one or more halo radicals;

More in particular, the invention relates to a pharmaceutical composition comprising a pharmaceutically acceptable carrier and, as active ingredients, an opioid analgesic and a therapeutically effective amount of a compound according to Formula (I), the pharmaceutically acceptable acid or base addition salts thereof, the stereochemically isomeric forms thereof, the N-oxide form thereof and a prodrug thereof, wherein R¹ is Ar¹methyl and attached to the 2-position or R¹ is Ar¹ and attached to the 3-position, as exemplified in either of the following formulas for compounds according to Formula (I) wherein m and n are equal to 1 and Ar is an unsubstituted phenyl. Preferably, Ar¹methyl is an unsubstituted benzyl radical. 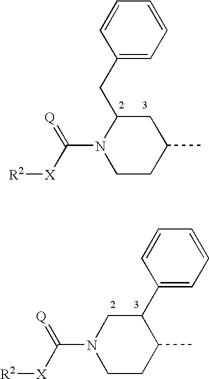
More in particular, the pharmaceutical composition comprises a compound according to the general Formula (I), the pharmaceutically acceptable acid or base addition salts thereof, the stereochemically isomeric forms thereof, the N-oxide form thereof and a prodrug thereof, wherein the R²—X—C(=Q)— moiety is 3,5-di-(trifluoromethyl) phenylcarbonyl.

More in particular, the pharmaceutical composition comprises a compound selected from the group of:

{4-[4-(1-benzoyl-piperidin-4-yl)-piperazin-1-yl]-2-benzyl-piperidin-1-yl}-(3,5-bis-trifluoromethyl-phenyl)-methanone; and

(2-benzyl-4-{4-[1-(4-methyl-[1,2,3]thiadiazole-5-carbonyl)-piperidin-4-yl]-piperazin-1-yl}-piperidin- I -yl)-(3,5-bis-trifluoromethyl-phenyl)-methanone.

Most in particular, the pharmaceutical composition comprises a compound according to Formula (I), the pharmaceutically acceptable acid or base addition salts thereof, the stereochemically isomeric forms thereof, the N-oxide form thereof and a prodrug thereof, with compound number 5, 110, 97, 45, 22, 151, 80, 62, 104, 8, 78, 12, 39, 113, 16, 56, 143, 36, 77, 106, 102, 6, 3, 142, 51, 9, 13, 32, 139, 4, 108, 89, 116, 2, 42, 140, 85, 37, 65, 133, 79, 64, 7, 141, 132, 134, 119, 90, 11, 26, 10 and 144 as cited in the Experimental section.

In the framework of this application, alkyl is defined as a monovalent straight or branched saturated hydrocarbon radical having from 1 to 6 carbon atoms, for example methyl, ethyl, propyl, butyl, 1-methylpropyl, 1,1-dimethylethyl, pentyl, hexyl; alkyl further defines a monovalent cyclic saturated hydrocarbon radical having from 3 to 6 carbon atoms, for example cyclopropyl, methylcyclopropyl, cyclobutyl, cyclopentyl and cyclohexyl. The definition of alkyl also comprises an alkyl radical that is optionally substituted on one or more carbon atoms with one or more phenyl, halo, cyano, oxo, hydroxy, formyl and amino radicals, for example hydroxyalkyl, in particular hydroxymethyl and hydroxyethyl and polyhaloalkyl, in particular difluoromethyl and trifluoromethyl.

In the framework of this application, halo is generic to fluoro, chloro, bromo and iodo.

In the framework of this application, with “compounds according to the invention” is meant a compound according to the general Formula (I), the pharmaceutically acceptable acid or base addition salts thereof, the stereochemically isomeric forms thereof, the N-oxide form thereof and a prodrug thereof.

In the framework of this application, especially in the moiety Alk^{a}-Y-Alk^{b} in Formula (I), when two or more consecutive elements of said moiety denote a covalent bond, then a single covalent bond is denoted. For example, when Alk^{a} and Y denote both a covalent bond and Alk^{b} is CH₂, then the moiety Alk^{a}-Y-Alk^{b} denotes —CH₂.

The pharmaceutically acceptable salts are defined to comprise the therapeutically active non-toxic acid addition salts forms that the compounds according to the invention are able to form. Said salts can be obtained by treating the base form of the compounds according to the invention with appropriate acids, for example inorganic acids, for example hydrohalic acid, in particular hydrochloric acid, hydrobromic acid, sulfuric acid, nitric acid and phosphoric acid; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclamic acid, salicylic acid, p-arninosalicylic acid and pamoic acid.

The compounds according to the invention containing acidic protons may also be converted into their therapeutically active non-toxic metal or amine addition salts forms by treatment with appropriate organic and inorganic bases. Appropriate base salts forms comprise, for example, the ammonium salts, the alkaline and earth alkaline metal salts, in particular lithium, sodium, potassium, magnesium and calcium salts, salts with organic bases, e.g. the benzathine, N-methyl-D-glucamine, hybramine salts, and salts with amino acids, for example arginine and lysine.

Conversely, said salt forms can be converted into the free forms by treatment with an appropriate base or acid.

The term addition salt as used in the framework of this application also comprises the solvates that the compounds according to the invention as well as the salts thereof, are able to form. Such solvates are, for example, hydrates and alcoholates.

The N-oxide forms of the compounds according to the invention are meant to comprise those compounds according to the invention wherein one or several nitrogen atoms are oxidized to the so-called N-oxide, particularly those N-oxides wherein one or more tertiary nitrogens (e.g. of the piperazinyl or piperidinyl radical) are N-oxidized. Such N-oxides can easily be obtained by a skilled person without any inventive skills and they are obvious alternatives for the compounds according to the invention since these compounds are metabolites, which are formed by oxidation in the human body upon uptake. As is generally known, oxidation is normally the first step involved in drug metabolism ( Textbook of Organic Medicinal and Pharmaceutical Chemistry, 1977, pages 70-75). As is also generally known, the metabolite form of a compound can also be administered to a human instead of the compound per se, with much the same effects.

The compounds according to the invention possess at least 2 oxydizable nitrogens (tertiary amines moieties). It is therefore highly likely that N-oxides are to form in the human metabolism.

The compounds according to Formula (I) may be converted to the corresponding N-oxide forms following art-known procedures for converting a trivalent nitrogen into its N-oxide form. Said N-oxidation reaction may generally be carried out by reacting the starting material according to Formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert-butyl hydroperoxide. Suitable solvents are, for example, water, lower alkanols, e.g. ethanol and the like, hydrocarbons, e.g. toluene, ketones, e.g. 2-butanone, halogenated hydrocarbons, e.g. dichloromethane, and mixtures of such solvents.

The term “stereochemically isomeric forms” as used hereinbefore defines all the possible isomeric forms that the compounds according to Formula (I) may possess. Unless otherwise mentioned or indicated, the chemical designation of compounds denotes the mixture of all possible stereochemically isomeric forms, said mixtures containing all diastereomers and enantiomers of the basic molecular structure. More in particular, stereogenic centers may have the R- or S-configuration; substituents on bivalent cyclic (partially) saturated radicals may have either the cis or trans-configuration. Compounds encompassing double bonds can have an E or Z-stereochemistry at said double bond. Stereochemically isomeric forms of the compounds according to Formula (I) are obviously intended to be embraced within the scope of this invention.

Following CAS nomenclature conventions, when two stercogenic centers of known absolute configuration are present in a molecule, an R or S descriptor is assigned (based on Cahn-Ingold-Prelog sequence rule) to the lowest-numbered chiral center, the reference center. The configuration of the second stereogenic center is indicated using relative descriptors [R*,R*] or [R*,S*], where R* is always specified as the reference center and [R*,R*] indicates centers with the same chirality and [R*,S*] indicates centers of unlike chirality. For example, if the lowest-numbered chiral center in the molecule has an S configuration and the second center is R, the stereo descriptor would be specified as S-[R*,S*]. If “α” and “β” are used: the position of the highest priority substituent on the asymmetric carbon atom in the ring system having the lowest ring number, is arbitrarily always in the “α” position of the mean plane determined by the ring system. The position of the highest priority substituent on the other asymmetric carbon atom in the ring system (hydrogen atom in compounds according to Formula (I)) relative to the position of the highest priority substituent on the reference atom is denominated “α”, if it is on the same side of the mean plane determined by the ring system, or “β”, if it is on the other side of the mean plane determined by the ring system.

Compounds according to the invention and some of the intermediate compounds have at least two stereogenic centers in their structure, namely at the 2- or 3-position of the piperidinyl-moiety (R and S) and at the 4-position, where the attached radical may be either in the cis or trans position with respect to the radical at the 2- or 3-position on the piperidinyl-moiety.

The invention also comprises pharmaceutical compositions according to the invention comprising derivative compounds (usually called “pro-drugs”) of the pharmacologically-active compounds according to the invention, which are degraded in vivo to yield the compounds according to the invention. Pro-drugs are usually (but not always) of lower potency at the target receptor than the compounds to which they are degraded. Pro-drugs are particularly useful when the desired compound has chemical or physical properties that make its administration difficult or inefficient. For example, the desired compound may be only poorly soluble, it may be poorly transported across the mucosal epithelium, or it may have an undesirably short plasma half-life. Further discussion on pro-drugs may be found in Stella, V. J. et al., “Prodrugs”, Drug Delivery Systms, 1985, pp. 112-176, and Drugs, 1985, 29, pp. 455-473.

Pro-drugs forms of the pharmacologically-active compounds according to the invention will generally be compounds according to the invention, having an acid group which is esterified or amidated. Included in such esterified acid groups are groups of the formula —COOR^{x}, where R^{x} is a C₁₋₆alkyl, phenyl, benzyl or one of the following groups 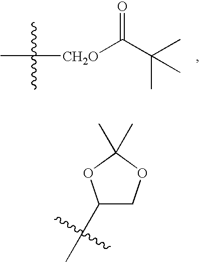
Amidated groups include groups of the formula —CONR^{y}R^{z}, wherein R^{y} is H, C₁₋₆alkyl, phenyl or benzyl and R^{z} is —H, —OH, C₁₋₆alkyl, phenyl or benzyl. Compounds according to the invention having an amino group may be derivatised with a ketone or an aldehyde such as formaldehyde to form a Mannich base. This base will hydrolyze with first order kinetics in aqueous solution.

The compounds according to Formula (I) as prepared in the processes described below may be synthesized in the form of racemic mixtures of enantiomers that can be separated from one another following art-known resolution procedures. The racemic compounds according to Formula (I) may be converted into the corresponding diastereomeric salt forms by reaction with a suitable chiral acid. Said diastereomeric salt forms are subsequently separated, for example, by selective or fractional crystallization and the enantiomers are liberated there from by alkali. An alternative manner of separating the enantiomeric forms of the compounds according to Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

In the framework of this application, the term opioid means opium-like or morphine-like in terms of pharmacological action. The broad group of opium alkaloids, synthetic derivatives related to the opium alkaloids, and the many naturally occuring and synthetic peptides with morphine-like pharmacological effects is called opioids. In addition to having pharmacological effects similar to those of morphine, a compound must be antagonized by an opioid antagonist such as naloxone to be classified as an opioid. The neuronally located proteins to which opioid agents bind to initiate a biological response are called opioid receptors. Opioids can act peripherally and centrally.

Suitable opioids or opioid analgesics for use in the present invention include one or more compounds selected from the group of alfentanil, buprenorphine, butorphanol, carfentanil, codeine, diacetylmorphine, dihydrocodeine, fentanyl, hydrocodone, hydromorphone, levorphanol, lofentanil, meperidine, methadone, morphine, nalbuphine, oxycodone, oxymorphone, pentazocine, propoxyphene, remifentanil and sufentanil; and pharmaceutical acceptable salts and derivatives thereof.

Because of their widespread use as analgesics, preferred opioid analgesics of use in the present invention are one or more compounds selected from the group of oxycodone, codeine, morphine, fentanyl, buprenorphine, hydrocodone, hydromorphone and pharmaceutical acceptable salts and derivatives thereof.

Suitable pharmaceutically acceptable salts of the opioid analgesics of use in the present invention include those salts described above in relation to the salts of the NK₁-antagonist.

Preferred salts of opioid analgesics of use in the present invention include alfentanil hydrochloride, buprenorphine hydrochloride, butorphanol tartrate, codeine phosphate, codeine sulphate, diacetylmorphine hydrochloride, dihydrocodeine bitartrate, fentanyl citrate, hydrocodone bitartrate, hydromorphone hydrochloride, levorphanol tartrate, meperidine hydrochloride, methadone hydrochloride, morphine sulphate, morphine hydochloride, morphine tartrate, nalbuphine hydrochloride, oxymorphone hydrochloride, pentazocine hydrochloride, propoxyphene hydrochloride and propoxyphene napsylate (2-naphthalene sulphonic acid (1:1) monohydrate).

Particular preferred opioid analgesics of use in the present invention are morphine, fentanyl and pharmaceutical acceptable salts and derivatives thereof.

More particular preferred opioid analgesics of use in the present invention are morphine sulphate and fentanyl citrate.

Pharmacology

The compounds according to the invention are potent inhibitors of neurokinin-mediated effects, in particular those mediated via the NK₁ receptor, and may therefore be described as neurokinin antagonists, especially as substance P antagonists, as indicated in vitro by the antagonism of substance P-induced relaxation of pig coronary arteries which is described hereinafter. The binding affinity of the present compounds for the human, guinea-pig and gerbil neurokinin receptors may be determined in vitro in a receptor binding test using ³H-substance-P as radioligand. The subject compounds also show substance-P antagonistic activity in vivo as may be evidenced by, for instance, the antagonism of substance P-induced plasma extravasation in guinea-pigs, or the antagonism of drug-induced emesis in ferrets (Watson et al., Br. J. Pharmacol. 115:84-94 (1995)).

The combination of an opioid analgesic with an NK₁ antagonist results in improved efficacy. Additional to the gain in efficacy, this combination also reduces several of the side-effects currently present with clinically used opioids. NK₁ receptor antagonists potentiating the analgesic activity of opioids require lower doses, resulting in a reduced risk of opioid side-effects, in particular emesis, respiratory depression and tolerance. But additionally it's seen that at similar doses (not lower opioid doses) there are also benefits of adding NK₁ to opioid.

Respiratory depression is the most serious side effect of opioid analgesics and is the primal cause of death from overdose. Opioids decrease the sensitivity of chemoreceptors in the brainstem to carbon dioxide, a normal stimulus of ventilatory reflexes. The result is a blunting of the ventilatory response to increases in the carbon dioxide tension (P_{co}_{₂}) in blood and cerebrospinal fluid. At equally effective analgesic doses, most opioids produce a similar degree of respiratory depression, as shown by an elevation in the blood P_{co}_{₂}. This effect is at least additive to that produced by other drugs that depress CNS functions, including general anesthetics and sedative-hypnotics. The mild respiratory depression produced by therapeutic doses of opioids is normally of little consequence. However, opioid analgesics must be used cautiously in patients with traumatic bead injuries, with emphysema and who are morbidly obese. At three to five times its usual analgesic dose, morphine can cause respiratory arrest in the nontolerant patient. In contrast, much higher doses will have minimal respiratory effects in morphine-tolerant individuals.

Tolerance refers to a reduced drug effect with repeated use and/or a need for higher doses to produce the same effect. Because tolerance does not occur to the same extent for all effects, drug abusers who take increasing amounts of drugs risk exposure to those effects to which tolerance does not develop. Tolerance develops to many of the effects of opioids. With repeated drug administration, larger doses are necessary to produce the same pharmacological response. The rate of tolerance development varies with the affected tissue of organ. Tolerance develops rapidly to the antiemetic effects of opioids; more gradually to their analgesic, endocrine and respiratory depressant effects; and virtually not at all to their constipating and miotic effects.

The compounds according to the invention have shown to reduce unwanted side-effects induced by opioids. Such reduction can be tested by in vivo testing using several species (e.g. ferrets, gerbils, rats, guinea pigs) and several pain models, covering pain models aiming at different states of acute and chronic pain, as well as animal models aiming to profile opioid side effects (such as opioid-induced emesis, GI transit and respiratory depression). For instance, the compounds of the present invention:
were able to inhibit the opioid-induced emesis in several species;
did not reduce the antinociceptive properties of opioids in models of acute, visceral and high intensity pain;
had an additive effect on the antinociceptive properties of opioids in models of inflammatory and chronic neuropathic pain;
reduced the respiratory depression induced by opioids in several species;
were able to reduce and overcome the tolerance observed with opioids daily administered in a model of chronic neuropatbic pain;
did not interfere with the discriminative central narcotic effects of opioids;
had no effect on the pharmacokinetics of opioids when administered concomitantly.
   This excludes pharmacokinetic interactions as the origin of the pharmacological effects observed.

The present invention therefore also relates to the use of a pharmaceutical composition according to the invention for the manufacture of a medicament for the prevention and/or treatment of pain and/or nociception.

In particular, the present invention relates to the use of a pharmaceutical composition according to the invention for the manufacture of a medicament for the opioid-based prevention and/or treatment of acute and chronic pain, more in particular in inflammatory, post-operative, emergency room (ER), breakthrough, neuropathic and cancer pain treatments.

The present invention further relates to the use of a pharmaceutical composition according to the invention for the manufacture of a medicament for the prevention and/or treatment of emesis in opioid-based treatments of pain.

The present invention further relates to the use of a pharmaceutical composition according to the invention for the manufacture of a medicament for the prevention and/or treatment of emesis in opioid-based treatments of pain, wherein the emesis is nausea and vomiting.

The present invention also relates to the use of an NK₁-receptor antagonist, in particular an NK₁-receptor antagonist according to Formula (I), the pharmaceutically acceptable acid or base addition salts thereof, the stereochemically isomeric forms thereof, the N-oxide form thereof and prodrugs thereof, for the manufacture of a medicament for the prevention and/or treatment of respiratory depression in opioid-based treatments of pain.

The present invention also relates to the use of an NK₁-receptor antagonist, in particular an NK₁-receptor antagonist according to Formula (I), the pharmaceutically acceptable acid or base addition salts thereof, the stereochemically isomeric forms thereof, the N-oxide form thereof and prodrugs thereof, for the manufacture of a medicament for reducing and/or overcoming the tolerance observed with opioids, e.g. when daily administered in chronic neuropathic pain.

To prepare the pharmaceutical compositions of this invention, an effective amount of the active ingredient, optionally in addition salt form, is combined in intimate admixture with a pharmaceutically acceptable carrier, which carrier may take a wide variety of forms depending on the form of preparation desired for administration. The pharmaceutical compositions are desirable in unitary dosage form suitable, in particular, for administration orally, rectally, percutaneously, by parenteral injection or by inhalation. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment. Other compositions may be compositions in a form suitable for sublingual, intranasal or pulmonary application or suitable as eye droplets.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

Since the compounds according to the invention are potent orally administrable NK₁ antagonists, pharmaceutical compositions comprising said compounds for administration orally are especially advantageous.

The NK₁-receptor antagonist and the opioid analgesic may be formulated in a single pharmaceutical product or composition or alternatively in individual pharmaceutical products or compositions for simultaneous, separate or sequential use in accordance with the present invention. The pharmaceutical product or composition may also be a product comprising the NK1-receptor antagonist and the opioid analgesic as separate unit dosages.

When administered in combination, either as a single or as separate pharmaceutical composition(s), the NK₁-receptor antagonist and the opioid analgesic are presented in a ratio which is consistent with the manifestation of the desired effect. In particular, the ratio by weight of the NK₁-antagonist to the opioid analgesic will suitably be approximately 1 to 1. Preferably, this ratio will be between 0.001 to 1 and 1000 to 1, and especially between 0.01 to 1 and 100 to 1.

A suitable dosage level for the NK₁-receptor antagonist is about 0.001 to 25 mg/kg per day, preferably about 0.005 to 10 mg/kg per day, and especially about 0.005 to 5 mg/kg day. The compounds may be administered on a regimen of up to 6 times per day, preferably 1 to 4 times per day.

The opioid analgesic may be administered at a dosage level up to conventional dosage levels for such analgesics, but preferably at a reduced level in accordance with the present invention. Suitable dosage levels will depend upon the analgesic effect of the chosen opioid analgesic, but typically suitable levels will be about 0.001 to 25 mg/kg per day, preferably 0.005 to 10 mg/kg per day, and especially 0.005 to 5 mg/kg day. The compound may be administered on a regimen of up to 6 times per day, preferably 1 to 4 times per day.

It will be appreciated that the amount of an NK₁-receptor antagonist and an opioid analgesic required for use in the prevention and/or treatment of pain and nociception will vary not only with the particular compounds or compositions selected but also with the route of administration, the nature of the condition being treated, and the age and condition of the human in need of such a treatment, and will ultimately be at the discretion of the attendant physician.

Preparation

The compounds according to the invention can generally be prepared by a succession of steps, each of which is known to the skilled person.

The compounds of Formula (I) are conveniently prepared by reductively N-alkylating an intermediate of Formula (II) wherein R¹, R², X, Q, m, n and p are defined as in Formula (I), with a N-substituted piperidinon of Formula (III) wherein R¹, Alk, Y, L and q are defined as in Formula (I). Said reductive N-alkylation may be performed in a reaction-inert solvent such as, for example, dichloromethane, ethanol or toluene or a mixture thereof, and in the presence of an appropriate reducing agent such as, for example, a botohydride, e.g. sodium borohydride, sodium cyanoborohydride or triacetoxy borohydride. In case a borohydride is used as a reducing agent, it may be convenient to use a complex-forming agent such as, for example, titanium(IV)-isopropylate as described in J. Org. Chem, 1990, 55, 2552-2554. Using said complex-forming agent may also result in an improved cis/trans ratio in favor of the trans isomer. It may also be convenient to use hydrogen as a reducing agent in combination with a suitable catalyst such as, for example, palladium-on-charcoal or platinum-on-charcoal. In case hydrogen is used as reducing agent, it may be advantageous to add a dehydrating agent to the reaction mixture such as, for example, aluminium tert-butoxide. In order to prevent the undesired further hydrogenation of certain functional groups in the reactants and the reaction products, it may also be advantageous to add an appropriate catalyst-poison to the reaction mixture, e.g., thiophene or quinoline-sulphur. Stirring and optionally elevated temperatures and/or pressure may enhance the rate of the reaction.

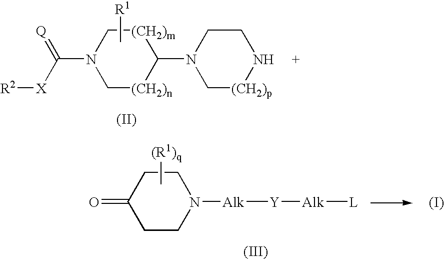

In this and the following preparations, the reaction products may be isolated from the reaction medium and, if necessary, further purified according to methodologies generally known in the art such as, for example, extraction, crystallization, trituration and chromatography.

Especially advantage is the preparation of a compound according to the invention according to the previous reaction scheme in which the Alk-Y-Alk-L-moiety is benzyl, thus giving rise to a compound according to Formula (I) in which the Alk-Y-Alk-L-moiety is benzyl. Said compound is pharmacological active and can be converted into a compound according to the invention in which the Alk-Y-Alk-L-moiety is hydrogen by reductive hydrogenation using e.g. hydrogen as a reducing agent in combination with a suitable catalyst such as, for example, palladium-on-charcoal or platinum-on-charcoal. The resulting compound according to the invention can then be converted into other compounds according to the invention by art-known transformations, e.g. acylation and alkylation.

In particular, the compounds of Formula (I^{a}) can be prepared by reacting a final compound of Formula (I¹) wherein R¹, R², X, Q, m, n, p and q are defined as in Formula (I) with an acyl compound of Formula (V) wherein Alk and L are defined as in Formula (I) and W¹ is an appropriate leaving group such as, for example, a halo, e.g. chloro or bromo, or a sulfonyloxy leaving group, e.g. methanesulfonyloxy or benzenesulfonyloxy. The reaction can be performed in a reaction-inert solvent such as, for example, a chlorinated hydrocarbon, e.g. dichloromethane, an alcohol, e.g. ethanol, or a ketone, e.g. methyl isobutylketone, and in the presence of a suitable base such as, for example, sodium carbonate, sodium hydrogen carbonate or triethylamine. Stirring may enhance the rate of the reaction. The reaction may conveniently be carried out at a temperature ranging between room temperature and reflux temperature.

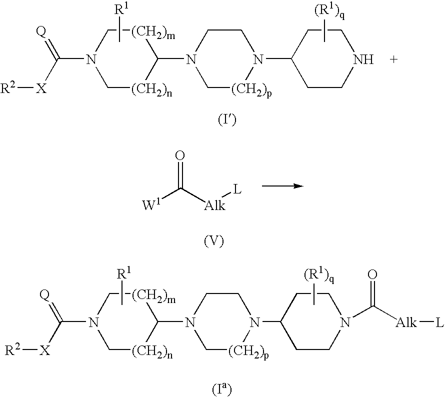

Alternatively, the compounds of Formula (I¹) can also be prepared by reacting a final compound of Formula (I¹) wherein R¹, R², X, Q, m, n, p and q are defined as in Formula (I) with a carboxylic acid of Formula (VI) wherein Alk and L are defined as in Formula (I)(base-catalyzed nucleophilic addition reaction). The reaction can be performed in a reaction-inert solvent such as, for example, a chlorinated hydrocarbon, e.g. dichloromethane, an alcohol, e.g. ethanol, or a ketone, e.g. methyl isobutylketone, and in the presence of a suitable base such as, for example, sodium carbonate, sodium hydrogen carbonate or triethylamine. Stirring may enhance the rate of the reaction. The reaction may conveniently be carried at a temperature ranging between room temperature and reflux temperature. 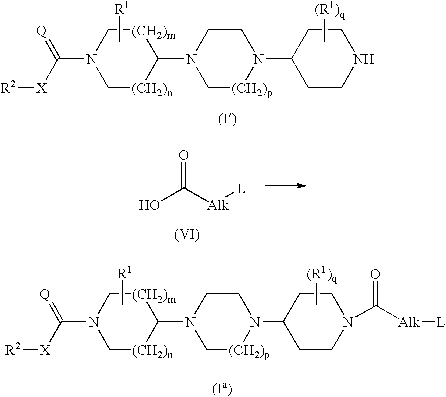
The above reaction may also be carried out under equivalent conditions with the carboxylic ester of the carboxylic acid of Formula (VI).

In particular, the compounds of Formula (I^{b}) can be prepared by reacting a final compound of Formula (I) wherein R¹, R², X, Q, m, n, p and q are defined as in Formula (I) with a keto-compound of Formula (VII) wherein W² is an appropriate leaving group such as, for example, a halogen, e.g. chloro or bromo, or a sulfonyloxy leaving group, e.g. methanesulfonyloxy or benzenesulfonyloxy. The reaction can be performed in a reaction-inert solvent such as, for example, a chlorinated hydrocarbon, e.g. dichloromethane, an alcohol, e.g. ethanol, or a ketone, e.g. methyl isobutylketone, and in the presence of a suitable base such as, for example, sodium carbonate, sodium hydrogen carbonate or triethylamine. Stirring may enhance the rate of the reaction. The reaction may conveniently be carried at a temperature ranging between room temperature and reflux temperature.

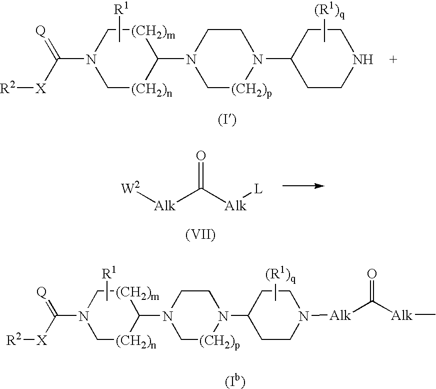

The compounds of Formula (I^{c}) can be prepared by reductive amination/alkylation of a final compound of Formula (I¹) wherein R¹, R², X, Q, m, n, p and q are defined as in Formula (I) with a compound of Formula (VIII) wherein Alk and L are defined as in Formula (I) and W³ is an appropriate leaving group such as, for example, a halogen, e.g. chloro or bromo, or a sulfonyloxy leaving group, e.g. methanesulfonyloxy or benzenesulfonyloxy. The reaction can be performed in a reaction-inert solvent such as, for example, a chlorinated hydrocarbon, e.g. dichloromethane, an alcohol, e.g. ethanol, or a ketone, e.g. methyl isobutylketone, and in the presence of a suitable base such as, for example, sodium carbonate, sodium hydrogen carbonate or triethylamine. Stirring may enhance the rate of the reactions The reaction may conveniently be carried at a temperature ranging between room temperature and reflux temperature.

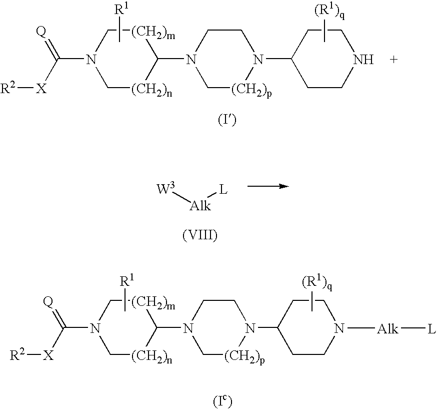

The starting materials and some of the intermediates are known compounds and are commercially available or may be prepared according to conventional reaction procedures generally known in the art. For example, intermediates of formula (II) may be prepared by reductively N-alkylating an intermediate of formula (IX) with an intermediate of formula (X) in which W⁴ is a benzyl radical, after which the compound according to Formula (X) is subsequently reduced to yield an intermediate compound according to Formula (II). Said reductive N-alkylation may be performed in a reaction-inert solvent such as, for example, dichloromethane, ethanol, toluene or a mixture thereof, and in the presence of an appropriate reducing agent such as, for example, a borohydride, e.g. sodium borohydride, sodium cyanoborohydride or triacetoxy borohydride. In case a borohydride is used as a reducing agent, it may be convenient to use a complex-forming agent such as, for example, titanium(IV)isopropylate as described in J. Org. Chem, 1990, 55, 2552-2554. Using said complex-forming agent may also result in an improved cis/trans ratio in favor of the trans isomer. It may also be convenient to use hydrogen as a reducing agent in combination with a suitable catalyst such as, for example, palladium-on-charcoal or platinum-on-charcoal. In case hydrogen is used as reducing agent, it may be advantageous to add a dehydrating agent to the reaction mixture such as, for example, aluminium tert-butoxide. In order to prevent the undesired further hydrogenation of certain functional groups in the reactants and the reaction products, it may also be advantageous to add an appropriate catalyst-poison to the reaction mixture, e.g., thiophene or quinoline-sulphur. Stirring and optionally elevated temperatures and/or pressure may enhance the rate of the reaction.

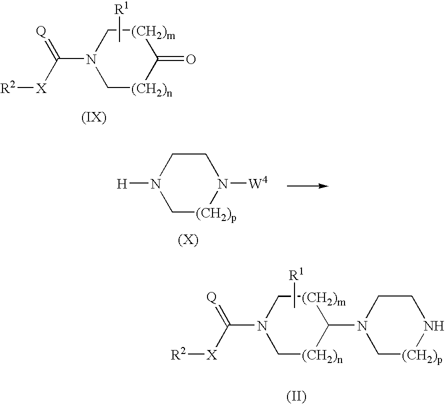

The preparation of these and other intermediates is described in WO 97/16440-A1, published May 9, 1997 by Janssen Pharmaceutica N.V, which is disclosed herein by reference as well as in other publications mentioned in WO 97/16440-A1, such as, e.g. EP-0,532,456-A and in our co-pending application WO 2004/033428 A1.

The following examples are intended to illustrate and not to limit the scope of the present invention.

### EXPERIMENTAL SECTION

Hereinafter “RT” means room temperature, “CDI” means 1,1′-carbonyldiimidazole, “DIPE” means diisopropylether, “MIK” means methyl isobutyl keton, “BINAP” means [1,1′-binaphthalene]-2,2′-diylbis[diphenylphosphinel, “NMP” means 1-methyl-2-pyrrolidinone, “Pd₂(dba)₃” means tris(dibenzylideneacetone)dipalladium and “DMF” means N,N-dimethylformamide.

### PREPARATION OF THE INTERMEDIATE COMPOUNDS

### Example A1

a. Preparation of Intermediate Compound 1 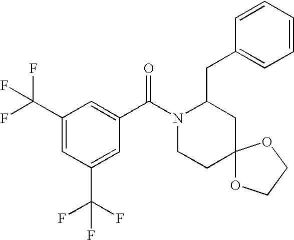
Et₃N (0.55 mol) was added to a stirring mixture of 7-(phenylmethyl)-1,4-dioxa-8-azaspiro[4.5]decane (0.5 mol) in toluene (1500 ml). 3,5-Bis(trifluoromethyl)benzoyl chloride (0.5 mol) was added over a 1-hour period (exothermic reaction). The mixture was stirred at room temperature for 2 hours, then allowed to stand for the weekend and washed three times with water (500 ml, 2×250 ml). The organic layer was separated, dried, filtered and the solvent was evaporated. Yielding: 245 g (100%). Part of this fraction was crystallized from petroleum ether. The precipitate was filtered off and dried. Yielding: 1.06 g of intermediate compound 1.
b. Preparation of Intermediate Compound 2 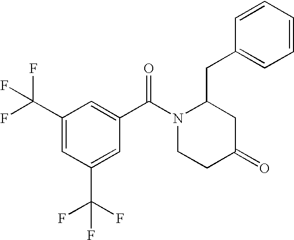
HCl cp (300 ml) was added to a mixture of intermediate compound 1 (0.5 mol) in ethanol (300 ml) and H₂O (300 ml). The reaction mixture was stirred at 60° C. for 20 hours. The precipitate was filtered off, ground, stirred in H₂O, filtered off, washed with petroleum ether and dried. Yielding: 192 g of intermediate compound 2 ((+−)-1-[3,5-bis(trifluoromethyl)benzoyl]-2-(phenylmethyl)-4-piperidinone) (89.4%) (mixture of R and S enantiomers).
c. Preparation of Intermediate Compound 3 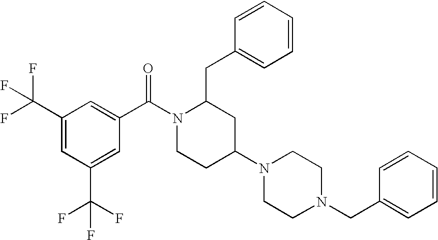
A mixture of intermediate compound 2 (0.046 mol), 1-(phenylmethyl)piperazine (0.051 mol) and C (0.056 mol) was stirred for 2 hours at 40° C. The reaction mixture was cooled to room temperature. Ethanol, p.a. (350 ml) was added. BH₄Na (0.138 mol) was added. The resulting reaction mixture was stirred for one hour at room temperature, then for one hour at 50° C. More BH₄Na (5.2 g) was added and the reaction mixture was stirred for 2 hours at 50° C. Again, BH₄Na was added and the reaction mixture was stirred overnight at room temperature, then for 2 hours at 50° C. Water (10 ml) was added. The mixture was stirred for 15 min. CH₂Cl₂ (200 ml) was added and the mixture was stirred for 15 min. The organic phase was separated, dried (MgSO₄), dicalite was added, the mixture was filtered over dicalite, and the filtrate was evaporated. This fraction was separated into (CIS) and (TRANS) by column chromatography over silica gel. The desired (TRANS)-fractions were collected and the solvent was evaporated, giving 14.8 g of residue ((I), 1.06 % (CIS)) and 4.9 g of residue ((II), 6% (CIS)). Resolution and purification of those (TRANS)-fractions (±20 g in total) was obtained by chromatography over stationary phase Chiralcel OD (1900Gr) in Prochrom LC110 35 bar (eluent: hexane/ethanol 90/10). The desired fractions were collected and the solvent was evaporated. Yielding: 9.5 g of intermediate compound 3 (2R-trans)-1-[3,5-bis(trifluoromethyl)benzoyl]-2-(phenylmethyl)-4-[4-(phenylmethyl)-1-piperazinyl]-piperidine.
d. Preparation of Intermediate Compound 4 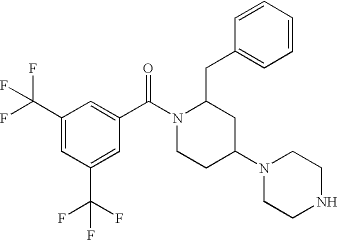
A mixture of intermediate compound 3 (0.288 mol) in methanol (700 ml) was hydrogenated at 40° C. with Pd/C, 10% (5 g) as a catalyst. After uptake of H₂ (1 eq), the catalyst was filtered off and the filtrate was evaporated. Yielding: 141.2 g of intermediate compound 4 (+)-(2R-trans)-1-[3,5-bis(trifluoromethyl)benzoyl]-2-(phenylmethyl)-4-(1-piperazinyl)piperidine.

### Example A2

Preparation of Intermediate Compound 5 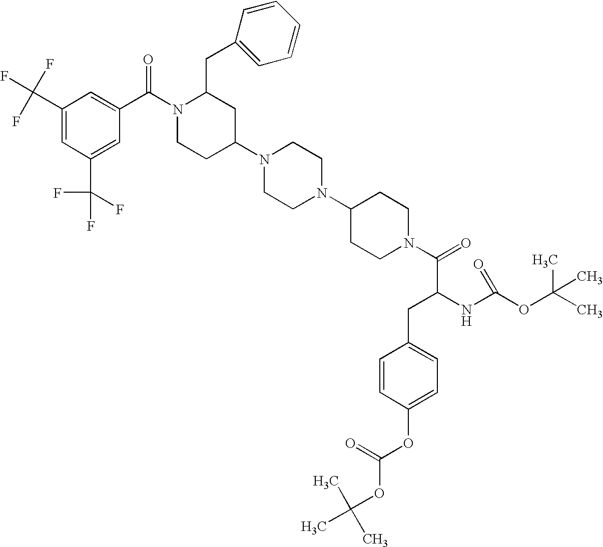
A mixture of N-[(1,1-dimethylethoxy)carbonyl]-L-tyrosine 1,1-dimethylcarbonate (0.005 mol), N,N-dimethyl-4-pyridinamine (0.006 mol) and Et₃N (0.006 mol) in CH₂Cl₂, p.a. (10 ml) was stirred at room temperature. N-(ethylcarbonimidoyl)-N,N-dimethyl-1,3-propanediamine monohydrochloride (0.006 mol) was added portionwise and was stirred for 45 minutes at room temperature. Then final compound 2 (described in example B1.b) (0.005 mol) was added and the reaction mixture was stirred overnight at room temperature. The mixture was washed with H₂O and Na₂CO₃. The separated organic layer was dried, filtered and the solvent was evaporated. The residue was purified over silica gel on a glass filter (eluent: CH₂Cl₂/MeHOH 100/0;98/2;96/4;94/6). The purest fractions were collected and the solvent was evaporated Yield: 1.4 g intermediate compound 5 (30%).

### Example A3

a. Preparation of Intermediate Compound 6 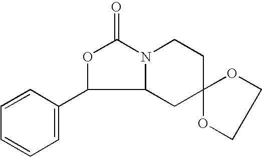
A mixture of 7-(hydroxyphenylmethyl)-1,4-dioxa-8-azaspiro[4,5]decane-8-carboxylic acid 1,1-dimethylethyl ester (0.5 mol) and 2-methyl-2-propanol potassium salt (6 g) in toluene (900 ml) was stirred and refluxed for 2 hours. The mixture was evaporated and the residue was stirred up in petrol ether and a little water. The mixture was decanted and the residue was stirred up in DIPE. The precipitate was filtered off and dried. Yielding: 127.4 g of intermediate compound 6 (92%).
b. Preparation of Intermediate Compound 7 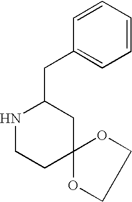
A mixture of intermediate compound 6 (0.5 mol) in methanol (700 ml) was hydrogenated at 50° C. overnight with Pd/C, 10% (5 g) as a catalyst. After uptake of H₂ (1 eq), the catalyst was filtered off and the filtrate was evaporated. The residue was taken up in water and extracted with CH₂Cl₂. The organic layer was dried (MgSO₄), filtered off and evaporated. Yielding: 99 g intermediate compound 7 (85%).
c. Preparation of Intermediate Compound 8 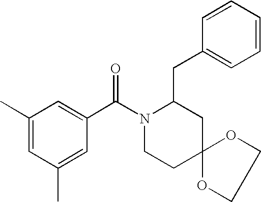
Et₃N (0.55 mol) was added to a mixture of intermediate compound 7 (0.5 mol) in toluene (1500 ml). 3,5-Dimethylbenzoyl chloride (0.5 mol) was added dropwise slowly over a 1-hour period while the temperature was kept below 50° C. and while stirring was continued. The mixture was stirred at room temperature overnight, then washed three times with water (500 ml, 2×250 ml) and separated into its layers. The organic layer was dried (MgSO₄), filtered and the solvent was evaporated. Yielding: 197 g (113%). Part of this fraction was dried. Yielding: 0.65 g of intermediate compound 8.
d. Preparation of Intermediate Compound 9 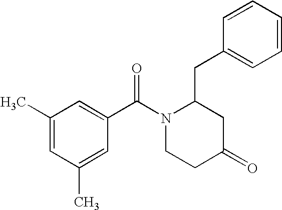
A mixture of intermediate compound 8 (0.56 mol) in ethanol (300 ml), HCl (300 ml) and H₂O (300 ml) was stirred at 60° C. for 8 hours. The mixture was stirred at room temperature for the weekend. The precipitate was filtered off, taken up in water, filtered off, washed with petroleum ether and dried. Yielding: 140.9 g of intermediate compound 9 (88%).
e. Preparation of Intermediate Compound 10 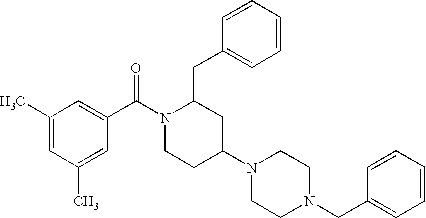
A mixture of intermediate compound 9 (0.05 mol) and 1-(phenylmethyl)-piperazine (0.05 mol) in thiophene, 4% solution (2 Ml) and toluene (500 ml) was hydrogenated with Pd/C, 10% (1 g) as a catalyst. After uptake of H₂ (1 eq), the catalyst was filtered off and the filtrate was evaporated. The residue was purified by column chromatography over silica gel (eluent: CH₂Cl₂/(CH₃OH/NH₃)99/1). The pure fractions were collected and evaporated. Yielding: 17.07 g (71%). The pure fractions of fraction 1 were collected and evaporated. Yielding: 2.5 g of intermediate compound 10 (10%).
f. Preparation of Intermediate Compound 11 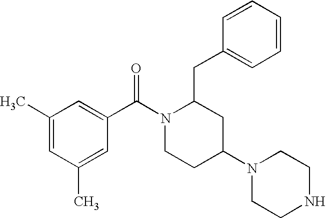
A mixture of intermediate compound 10 (0.0052 mol) in methanol (100 ml) was hydrogenated at 50° C. for one night with Pd/C, 10% (1 g) as a catalyst. After uptake of H₂ (1 eq), the catalyst was filtered off and the filtrate was evaporated. The residue was purified on a glass filter over silica gel (eluent: CH₂Cl₂/(CH₃OH/NH₃NT₃) 99/1, 98/2, 97/3, 96/4 and 95/5). The pure fractions were collected and evaporated. Yielding: 1.7 g on intermediate compound 11 (83%).

### Example A4

Preparation of Intermediate Compound 12 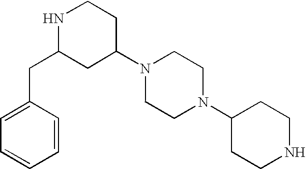
A mixture of final compound 2 (prepared according to B1b) (0.01 mol) and KOH (0.15 mol) in 2-propanol (50 ml) was stirred and refluxed for 18 hours. The solvent was evaporated, then the residue was taken up in H₂O (20 ml) and the mixture was extracted with CH₂Cl₂. The organic layer was washed with NaOH (1 N), dried (MgSO₄), filtered and the solvent was evaporated. Yield: 3.25 g of intermediate compound 12 (95%).

### PREPARATION OF THE FINAL COMPOUNDS

### Example B1

a. Preparation of Final Compound 1 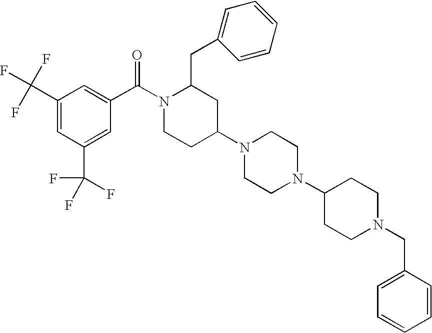
A mixture of intermediate compound 4 (0.12 mol) and 1-(phenylmethyl)-4-piperidinone (0.12 mol) in methanol (250 ml) was hydrogenated (H1163-066) at 50° C. with Pd/C 10% (3 g) as a catalyst in the presence of thiophene solution (2 ml). After uptake of H₂ (1 eq), the catalyst was filtered off and the filtrate was evaporated. The residue was suspended in petroleum ether, filtered off and crystallized from DIPE. Yield: 46 g (F1). The filtrate was evaporated. Yield: 37.7 g (F2). F1 and F2 were combined and purified by column chromatography over silica gel (eluent: CH₂Cl₂/MeOH 91/9). The product fractions were collected and the solvent was evaporated. Yield: 46 g (F3). F3 was crystallized from DIPE. Yield: 0.65 g of final compound 1.
b. Preparation of Final Compound 2 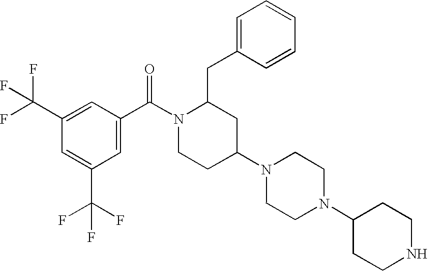
A mixture of final compound 1 (0.0074 mol) in methanol (150 ml) was hydrogenated (H163-077) with Pd/C 10% (1 g) as a catalyst. After uptake of H₂ (1 eq), the catalyst was filtered off and the filtrate was concentrated. Yield: 4.3 g of final compound 2.

### Example B2

Preparation of Final Compound 3 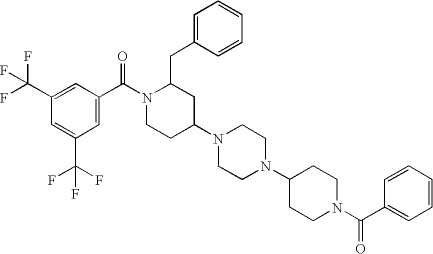
A mixture of compound 2 (0.0015 mol) and Et₃N (0.1 mol) in CH₂Cl₂ (100 ml) was stirred at room temperature. Benzoylchloride (0.0025 mol) was dissolved in CH₂Cl₂ and added dropwise to the reaction mixture. The mixture was stirred for 1 hour at room temperature. NaOH (1 N; 100 ml) was added and the mixture was stirred for 30 minutes at room temperature. The separated aqueous layer was extracted with CH₂Cl₂. The organic layer was washed with H₂O, dried (MgSO₄), filtered and the solvent was evaporated. The residue was purified by column chromatography over silica gel (eluent: CH₂Cl₂/MeOH 100/0;90/10). The desired fractions were collected and the solvent was evaporated. Yield: 0.624 g of final compound 3. (61%).

### Example B3

a. Preparation of Final Compound 4 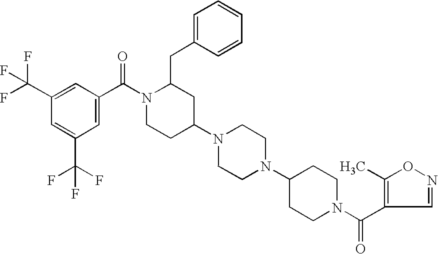
A mixture of 5-methyl4-isoxazolecarboxylic acid (0.0015 mol) in CH₂Cl₂ (20 ml) and 1,1′-carbonylbis-1H-imidazole (0.0015 mol) was stirred for 2 hours at room temperature. Compound 2 (prepared according to B1.b) (0.001 mol) was added. After stirring overnight, the reaction mixture was washed with diluted NaOH, washed with H₂O, dried, filtered and the solvent evaporated. The residue was purified by column chromatography over silica gel (eluent: CH₂Cl₂-gradient 0->10% MeOH). The product fractions were collected and the solvent evaporated. The residue was dried. Yield: 0.204 g of final compound 4.
b. Preparation of Final Compound 5 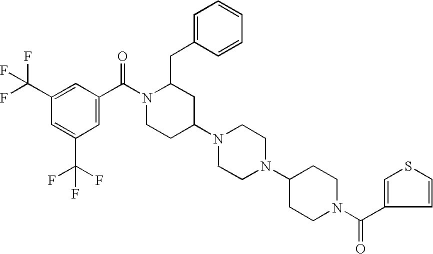
A mixture of 3-thiophenecarboxylic acid (0.00188 mol), N,N-dimethyl-4-pyridinamine (0.00255 mol) and Et₃N (0.00255 mol) in CH₂Cl₂ (200 ml) was stirred at room temperature. N,N-dimethyl-N′-(methylcarbonimidoyl)-1,3-propanediamine (0.00255 mol) was added portionwise and the mixture was stirred for one hour at room temperature. A solution of compound 2 (prepared according to B1b) (0.00188 mol) in CH₂Cl₂ was added dropwise and the reaction mixture was stirred over the weekend at room temperature. The mixture was poured out into 1 g NaOH/water. The layers were separated. The water layer was extracted with CH₂Cl₂. The separated organic layer was dried (MgSO₄), filtered and the solvent evaporated. The residue was purified by column chromatography over silica gel (eluent: CH₂Cl₂/CH₃OH from 100/0 to 90/10). The product fractions were collected and the solvent was evaporated. Yield: 0.749 g of compound 5 (58%).

### Example B4

a. Preparation of Final Compound 6 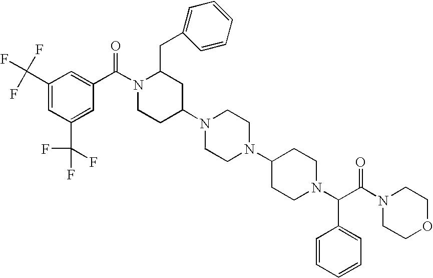
A mixture of compound 2 (prepared according to B1b) (0.005 mol), 4(chlorophenyl-acetyl)-morpholine (0.005 mol) and Na₂CO₃ (0.01 mol) in MIK, p.a. (125 ml) was stirred and refluxed for 18 hours using a water separator. The reaction mixture was washed with water, dried, filtered and the solvent evaporated. The residue was purified over silica gel on a glass filter (eluent: CH₂Cl₂/(CH₃OH/NH₃) 95/5). The product fractions were collected and the solvent was evaporated. The residue was suspended in DIPE, filtered off and dried. Yield: 1.702 g of compound 6.
b. Preparation of Final Compound 7 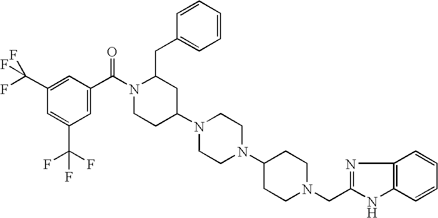
A mixture of compound 2 (prepared according to B1b) (0.0012 mol), 2-(chloromethyl)-1H-benzimidazole (0.0014 mol) and K₂CO₃ (0.0018 mol) in CH₃CN (5 ml) was stirred and refluxed for 12 hours, then cooled to room temperature and the solvent was evaporated. The residue was taken up in CH₂Cl₂. The organic layer was washed with H₂O, dried (MgSO₄), filtered and the solvent was evaporated. The residue (0.95 g) was purified by column chromatography over silica gel (eluent: CH₂Cl₂/CH₃OH/NH₄OH 90/10/0.5; 15-40 μm). The pure fractions were collected and the solvent was evaporated. The residue (0.14 g) was crystallized from DIPE. The precipitate was filtered off and dried. Yielding: 0.087 g of compound 7 (10%) (mp. 135° C.).
c. Preparation of Final Compound 8 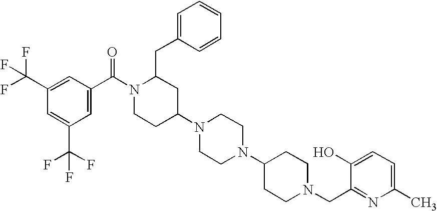
A mixture of compound 2 (prepared according to B1b) (0.005 mol) and 2-(chloromethyl)-6-methyl-3-pyridinol (0.006 mol) was taken up in DMF (50 ml). N-methyl-N-(1-methylethyl)-propanamine (0.02 mol) was added. The reaction mixture was stirred overnight at ±65° C. The solvent was evaporated. The residue was taken up in CH₂Cl₂ and washed with a diluted NH₃ solution. The separated organic layer was dried, filtered and the solvent was evaporated. The residue was purified by column chromatography over silica gel (eluent: CH₂Cl₂/(MeO₄/NH₃) 95/5). The desired fractions were collected and the solvent was evaporated. The residue was suspended in DIPE. The precipitate was filtered off and dried. Yield: 1.423 g of compound 8.

### Example B5

Preparation of Final Compound 9 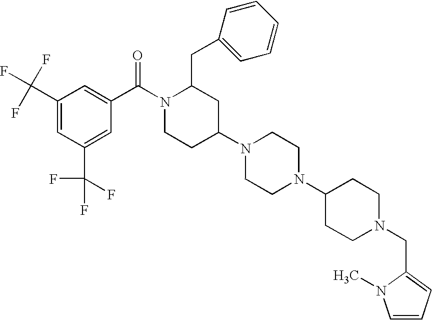
A mixture of compound 2 (prepared according to B1b) (0.003 mol) and 1-methyl-1H-pyrrole-2-carboxaldehyde (0.0046 mol) was hydrogenated at 50° C. under H₂ with Pd/C 10% (1 g) as a catalyst in the presence of thiophene solution (1 ml). After uptake of H₂ (1 eq), the catalyst was filtered off and the filtrate was evaporated. The residue was purified by column chromatography over silica gel (eluent: CH₂C₂/(MeOH/NH₃) 97/3;95/5). The product fractions were collected and the solvent was evaporated. The residue was suspended in petroleumether. Yield: 1.079 g of compound 9.

### Example B6

Preparation of Final Compound 10 and 11 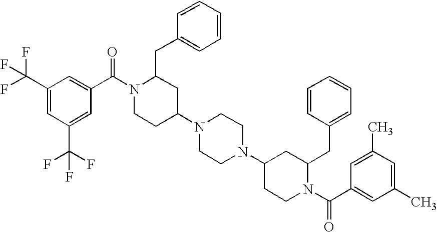
[2α,4α(2R*,4S*)]=compound 10
(2α, 4β(2R*,4S*)]=compound 11
A mixture of intermediate compound 2 (prepared according to A1b) (0.005 mol), intermediate compound 11 (prepared according to A3f) (0.005 mol) and Ti(OiPro)₄ (3 g) in methanol (150 ml) was hydrogenated at 50° C. under N₂ flow with Pd/C 10% (1 g) as a catalyst in the presence of thiophene solution (1 ml). After uptake of H₂ (1 eq), the catalyst was filtered off and the filtrate was evaporated. The residue was taken up in H₂O and CH₂Cl₂. The mixture was stirred for 10 min and filtered over dicalite. The organic layer was separated, dried, filtered and the solvent was evaporated. The residue was purified by column chromatography over silica gel (eluent: CH₂Cl₂(CH₃OH/NH₃) 97/3). Two fractions were collected and their solvents were evaporated. Yielding: 0.53 g compound 10 and 0.4 g of compound 11.

### Example B7

Preparation of Final Compound 12 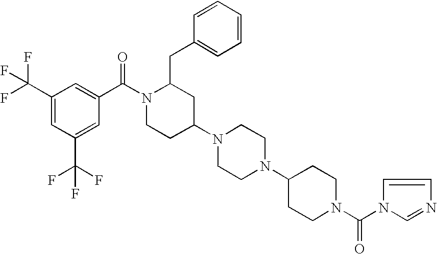
A mixture of compound 2 (prepared according to B1b) (0.001 mol) in CH₂Cl₂ (50 ml) and C (0.0015 mol) was stirred overnight. The reaction mixture was washed with diluted NaOH, washed with H₂O, dried and the solvent was evaporated. The residue was purified by column chromatography over silica gel (Eluent: CH₂Cl₂/CH₃OH 100/0 and 90/10). The product fractions were collected and the solvent evaporated. Yield: 0.645 g of compound 12.

### Example B8

Preparation of Final Compound 13 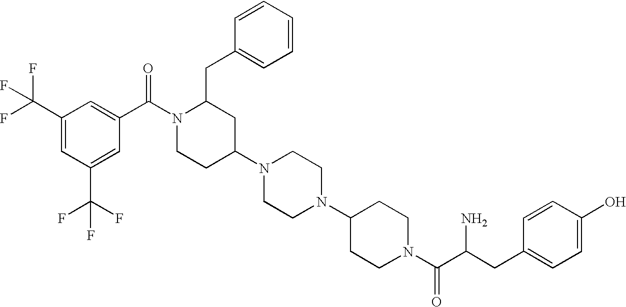
A mixture of intermediate compound 12 (prepared according to A4) (0.0015 mol) in HCl/2-propanol (5 ml) and methanol (20 ml) was stirred and refluxed for 1 hour. The reaction mixture was crystallized, filtered off and dried. Yield: 0.43 g of final compound 13 (38%)

### Example B9

Preparation of Final Compound 40 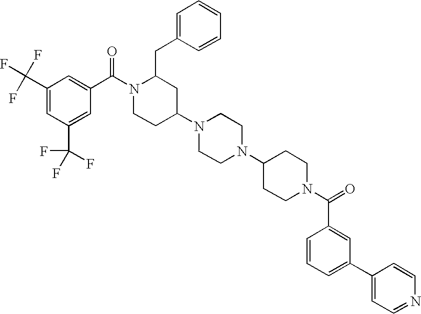
A mixture of final compound 31 (prepared according to B2)(0.065 mmol), 4-pyridinyl-boronic acid (0.09 mmol), Pd(OAc)₂ (0.015 mmol), 1,3-bis(diphenylphosphino)propane (0.03 mmol), Na₂CO₃, 2M (1 ml) and DME (2 ml) was stirred at 100° C. for 16 hours. The solvent was evaporated and the residue was taken up in H₂O and extracted with CH₂Cl₂. The organic layer was separated, dried with MgSO₄ and the solvent evaporated. The residue was purified by column chromatography over kromasil (gradient: CH₂Cl₂/CH₃OH 95/5). The desired fractions were collected and the solvent was evaporated. Yield: 1 mg of final compound 40.

### Example B10

Preparation of Final Compound 85 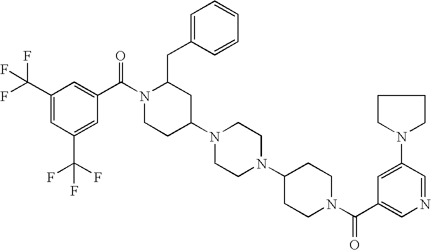
A mixture of final compound 83 (prepared according to B2)(0.0004 mol), pyrrolidine (0.0006 mol), Pd₂(dba)₃ (0.00001 mol), BINAP (0.00003 mol) and 2-methyl-2-propanol sodium salt (0.0006 mol) in toluene (5 ml) was stirred at 100° C. for 16 hours. The solvent was evaporated and the residue was taken up in H₂O and extracted with CH₂Cl₂. The organic layer was separated, dried with MgSO₄ and the solvent evaporated. The residue was purified by column chromatography over kromasil (gradient: CH₂Cl₂/CH₃OH 95/5). The desired factions were collected and the solvent was evaporated. Yield: 0.119 g of final compound 85.

### Example B11

Preparation of Final Compound 43 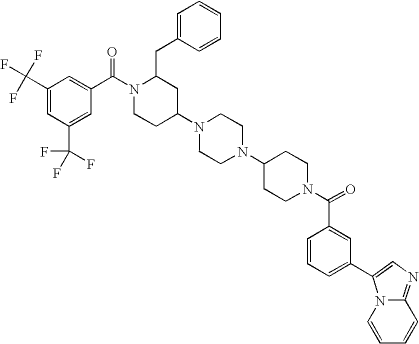
A mixture of final compound 31 (prepared according to B2)(0.065 mmol), imidazo(1,2-a)pyridine (0.09 mmol), Pd(OAc)₂ (0.015 mmol), 1,3-bis(diphenyl-pbosphino)propane (0.03 mmol) and Cs₂CO₃ (0.09 mmol) in NMP (5 ml) was stirred at 140° C. for 16 hours. The solvent was evaporated and the residue was taken up in H₂O and extracted with CH₂Cl₂. The organic layer was separated, dried with MgSO₄ and the solvent evaporated. The residue was purified by column chromatography over kromasil (gradient: CH₂Cl₂/CH₃OH 95/5). The desired factions were collected and the solvent was evaporated. The desired fractions were collected and the solvent was evaporated. Yield: 8 mg of final compound 43.

### Example B12

Preparation of Final Compound 44 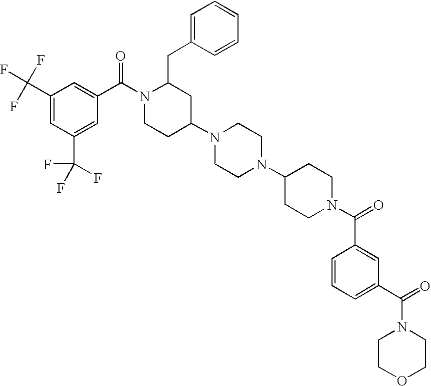
A mixture of compound 31 (prepared according to B2)(0.065 mmol), morpholine (0.2 mmol), Pd(OAc)₂ (0.015 mmol) and 1,3-bis(diphenylphosphino)-propane (0.03 mmol) in diglyme (3 ml) under 1 atmosphere CO was stirred at 150° C. for 16 hours. The solvent was evaporated and the residue was taken up in H₂O and extracted with CH₂Cl₂. The organic layer was separated, dried with MgSO₄ and the solvent evaporated. The residue was purified by column chromatography over kromasil (gradient: CH₂Cl₂/CH₃OH 95/5). The desired fractions were collected and the solvent was evaporated. The desired fractions were collected and the solvent was evaporated. Yield: 3 mg of final compound 44.

### Example B13

Preparation of Final Compound 144 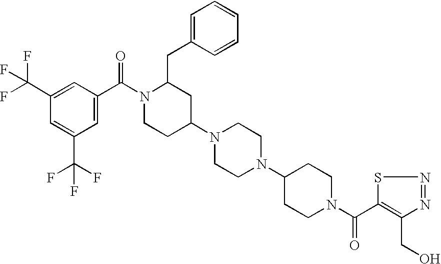
A mixture of 4-[(4-acetyloxy)methyl]-1,2,3-thiadiazole-5-carboxylic acid methyl ester (0.001 mol), final compound 2 (prepared according to B1b) (0.002 mol), NaCN (20 mg) in methanol (20 ml) was stirred and refluxed for 20 hours. The solvent was evaporated and the residue was purified by column chromatography over silica gel (eluent: CH₂Cl₂/MeOH from 100/0 to 80/20). The desired fractions were collected and the solvent was evaporated. The residue was suspended in petroleum ether. The precipitate was filtered off and dried. Yield: 0.110 g of final compound 144.

### Example B14

Preparation of Final Compound 130 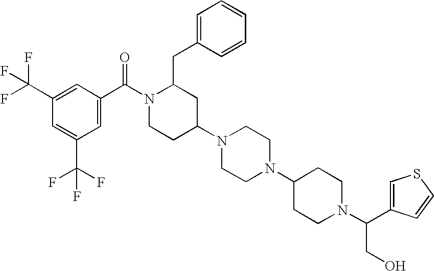
A mixture of final compound 2 (prepared according to B1b) (0.001 mol), glycolaldehyde dimer (0.001 mol) and 3-thiophene boronic acid (0.001 mol) in 2,2,2-trifluoroethanol (5 ml) was stirred at room temperature for 18 hours. This was followed by addition of a solution of K₂CO₃ (10%) and extraction with ethyl acetate. The combined organic layers were dried (MgSO₄), filtered and concentrated under vacuum. The residue (0.6 g) was purified by chromatography on a silicagel column (CH₂Cl₂/MeOH/NH₄OH 92/08/0.2) and the product fractions were concentrated, providing 0.29 g (47%) of final compound 130.

### Example B15

Preparation of Final Compound 153 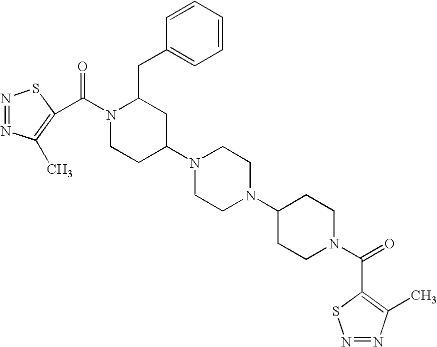
A mixture of intermediate compound 12 (prepared according to A4) (0.00934 mol) and Et₃N (0.02 mol) in CH₂Cl₂ (200 ml) was stirred on an ice bath, then a solution of 4-methyl-1,2,3-thiadiazole-5-carbonyl chloride (0.00943 mol) in CH₂Cl₂ (20 ml) was added dropwise over 15 minutes at 0° C. The reaction mixture allowed to reach room temperature and was stirred for 1 hour at room temperature, NaOH (20 ml) was added and the reaction mixture was stirred for 15 minutes at room temperature. The layers were separated and the aqueous layer was extracted with CH₂Cl₂. The organic layer was washed with H₂O, dried (MgSO₄), filtered off and the solvent was evaporated. The residue was purified by column chromatography over silicagel (eluent: CH₂Cl₂/MeOH/(MeOH/NH₃) from 100/0/0 to 90/10/0 to 90/1 0/0). Two product fractions were collected and each solvent was evaporated. Yield fraction 1:1.260 g of final compound 153 (22%).

The compounds exemplified in the following Tables 1-5 were prepared in a manner analogous to one of the foregoing examples B1 to B15.

TABLE 1

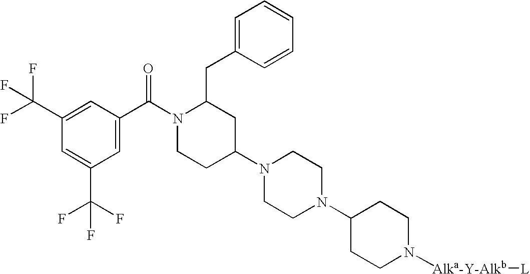

| Comp. | Exp. | | | | | Physical |
|---|---|---|---|---|---|---|
| No. | No. | Alk^{a} | Y | Alk^{b} | L | data |
| 2 | B1b | cb | cb | cb | H | 2R-trans |
| 121 | B1b | cb | cb | cb | H | 2R-cis |
| 122 | B1b | cb | cb | cb | H | 2S-trans |
| 123 | B1b | cb | cb | cb | H | 2S-cis |
| 15 | B4b | cb | cb | cb | 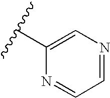 | 2R-trans |
| 16 | B4a | cb | cb | cb | 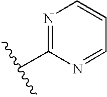 | 2R-trans |
| 17 | B4c | cb | cb | cb | 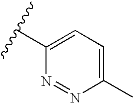 | 2R-trans |
| 18 | B4c | cb | cb | cb | 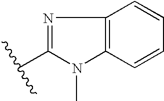 | 2R-trans |
| 124 | B4c | cb | cb | cb | 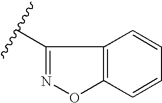 | 2R-trans |
| 9 | B5 | —CH₂— | cb | cb | 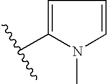 | 2R-trans |
| 20 | B4b | —CH₂— | cb | cb | 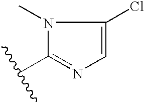 | 2R-trans |
| 8 | B4c | —CH₂— | cb | cb | 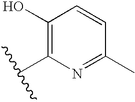 | 2R-trans |
| 7 | B4b | —CH₂— | cb | cb | 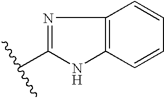 | 2R-trans |
| 21 | B4b | —CH₂— | cb | cb | 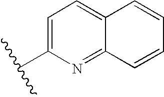 | B-trans |
| 125 | B1a | —CH₂— | cb | cb | 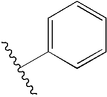 | 2R-cis |
| 126 | B1a | —CH₂— | cb | cb | 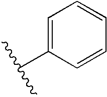 | 2S-cis |
| 1 | B1a | —CH₂— | cb | cb | 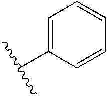 | 2R-trans |
| 127 | B1a | —CH₂— | cb | cb | 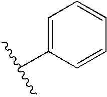 | 2S-trans |
| 22 | B4b | —CH₂— | cb | cb | 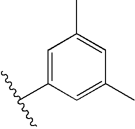 | 2R trans |
| 23 | B4b | —CH₂— | cb | cb | 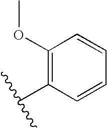 | 2R-trans |
| 24 | B4b | —CH₂— | cb | cb | 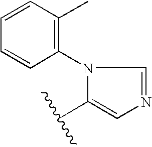 | 2R-trans |
| 25 | B4b | —CH₂— | cb | cb | 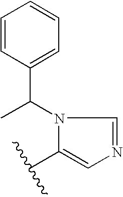 | B-trans |
| 26 | B4b | —CH₂— | cb | cb | 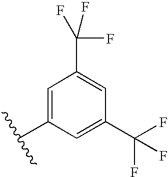 | B-trans |
| 27 | B4b | —CH₂—CH═CH— | cb | cb | 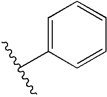 | [2B-[2α,4β(E)]] |
| 128 | B14 | 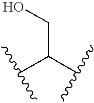 | cb | cb | 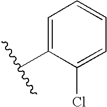 | 2R-trans |
| 129 | B14 | 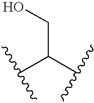 | cb | cb | 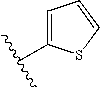 | 2R-trans |
| 130 | B14 | 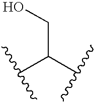 | cb | cb | 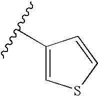 | 2R-trans |
| 131 | B14 | 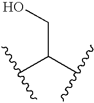 | cb | cb | 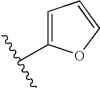 | 2R-trans |
| 28 | B4c | 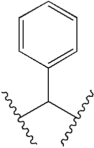 | cb | cb | 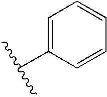 | B-trans |
| 29 | B2 | cb | C═O | cb | 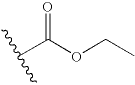 | 2R-trans |
| 162 | B3b | cb | C═O | cb | 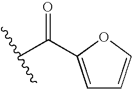 | 2R-trans |
| 30 | B2 | cb | C═O | cb | 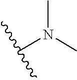 | 2R-trans |
| 3 | B2 | cb | C═O | cb | 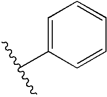 | 2R-trans mp. 142.5° C. |
| 132 | B2 | cb | C═O | cb | 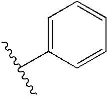 | 2S-trans |
| 133 | B2 | cb | C═O | cb | 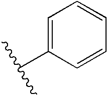 | 2R-cis |
| 134 | B2 | cb | C═O | cb | 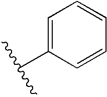 | 2S-cis |
| 31 | B2 | cb | C═O | cb | 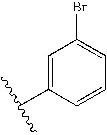 | 2R-trans |
| 32 | 132 | cb | C═O | cb | 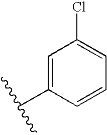 | 2R-trans |
| 165 | B2 | cb | C═O | cb | 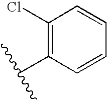 | 2R-trans |
| 33 | B2 | cb | C═O | cb | 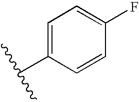 | 2R-trans |
| 34 | B2 | cb | C═O | cb | 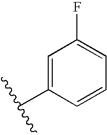 | 2R-trans |
| 164 | B2 | cb | C═O | cb | 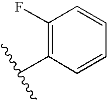 | 2R-trans |
| 35 | B3b | cb | C═O | cb | 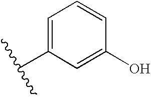 | 2R-trans |
| 36 | B2 | cb | C═O | cb | 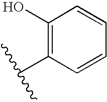 | 2R-trans |
| 163 | B2 | cb | C═O | cb | 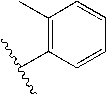 | 2R-trans |
| 37 | B2 | cb | C═O | cb | 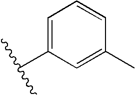 | 2R-trans |
| 135 | B2 | cb | C═O | cb | 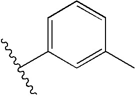 | 2R-trans HCl(1:2) |
| 38 | B2 | cb | C═O | cb | 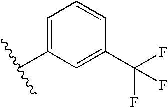 | 2R-trans |
| 39 | B3a | cb | C═O | cb | 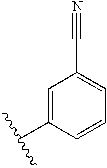 | 2R-trans |
| 40 | B9 | cb | C═O | cb | 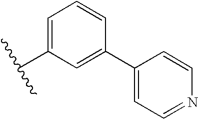 | 2R-trans |
| 41 | B10 | cb | C═O | cb | 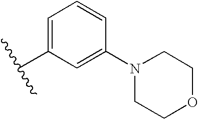 | 2R-trans |
| 42 | B10 | cb | C═O | cb | 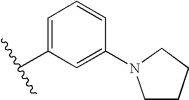 | 2R-trans |
| 43 | B11 | cb | C═O | cb | 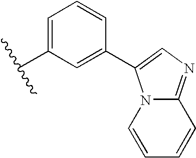 | 2R-trans |
| 44 | B12 | cb | C═O | cb | 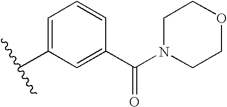 | 2R-trans |
| 45 | B12 | cb | C═O | cb | 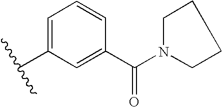 | 2R-trans |
| 46 | B2 | cb | C═O | cb | 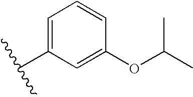 | B-trans |
| 47 | B2 | cb | C═O | cb | 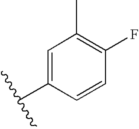 | 2R-trans |
| 48 | B2 | cb | C═O | cb | 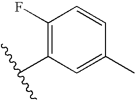 | 2R-trans |
| 49 | B2 | cb | C═O | cb | 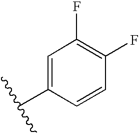 | 2R-trans |
| 50 | B2 | cb | C═O | cb | 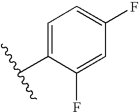 | 2R-trans |
| 51 | B2 | cb | C═O | cb | 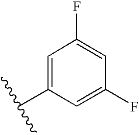 | 2R-trans |
| 52 | B2 | cb | C═O | cb | 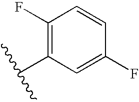 | 2R-trans |
| 53 | B2 | cb | C═O | cb | 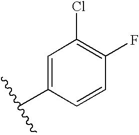 | 2R-trans |
| 54 | B2 | cb | C═O | cb | 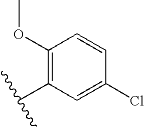 | 2R-trans |
| 55 | B2 | cb | C═O | cb | 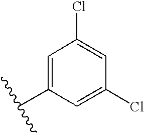 | 2R-trans |
| 56 | B3b | cb | C═O | cb | 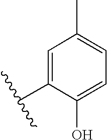 | 2R-trans |
| 57 | B2 | cb | C═O | cb | 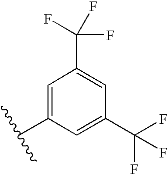 | B-trans |
| 58 | B2 | cb | C═O | cb | 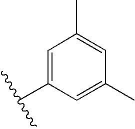 | 2R-cis |
| 59 | B2 | cb | C═O | cb | 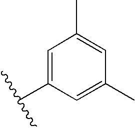 | B-trans |
| 60 | B2 | cb | C═O | cb | 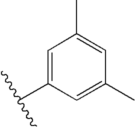 | trans |
| 170 | B3b | cb | C═O | cb | 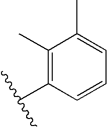 | 2R-trans |
| 61 | B2 | cb | C═O | cb | 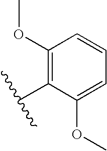 | 2R-trans |
| 62 | B2 | cb | C═O | cb | 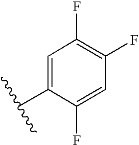 | 2R-trans |
| 63 | B2 | cb | C═O | cb | 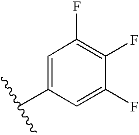 | 2R-trans |
| 64 | B3a | cb | C═O | cb | 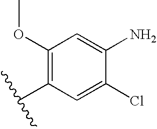 | 2R-trans |
| 65 | B2 | cb | C═O | cb | 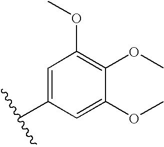 | 2R-trans |
| 66 | B2 | cb | C═O | cb | 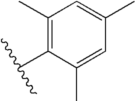 | B-trans |
| 67 | B3b | cb | C═O | cb | 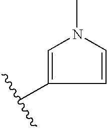 | 2R-trans |
| 68 | B2 | cb | C═O | cb | 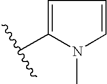 | 2R-trans |
| 69 | B3a | cb | C═O | cb | 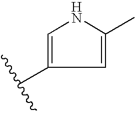 | 2R-trans |
| 5 | B3b | cb | C═O | cb | 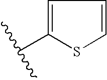 | 2R-trans |
| 70 | B2 | cb | C═O | cb | 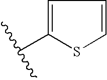 | 2R-trans |
| 161 | B2 | cb | C═O | cb | 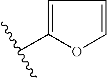 | 2R-trans |
| 71 | B3a | cb | C═O | cb | 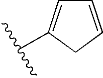 | 2R-trans |
| 136 | B3b | cb | C═O | cb | 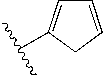 | 2S-trans |
| 137 | B3b | cb | C═O | cb | 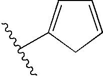 | 2R-cis |
| 138 | B3b | cb | C═O | cb | 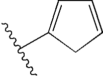 | 2S-cis |
| 72 | B3a | cb | C═O | cb | 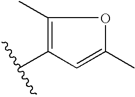 | 2R-trans |
| 12 | B7 | cb | C═O | cb | 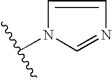 | 2R-trans |
| 73 | B2 | cb | C═O | cb | 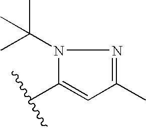 | 2R-trans |
| 19 | B2 | cb | C═O | cb | 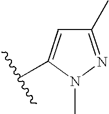 | 2R-trans |
| 74 | B2 | cb | C═O | cb | 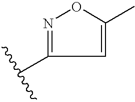 | 2R-trans |
| 75 | B2 | cb | C═O | cb | 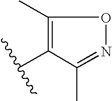 | 2R-trans |
| 4 | B3a | cb | C═O | cb | 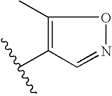 | 2R-trans |
| 76 | B3a | cb | C═O | cb | 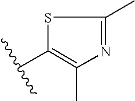 | 2R-trans |
| 77 | B2 | cb | C═O | cb | 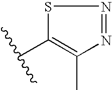 | 2R-trans m.p. 119.6° C. |
| 139 | B2 | cb | C═O | cb | 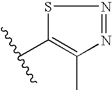 | 2R-cis |
| 140 | B2 | cb | C═O | cb | 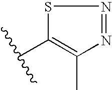 | 2S-cis |
| 141 | B2 | cb | C═O | cb | 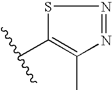 | 2S-trans |
| 78 | B2 | cb | C═O | cb | 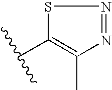 | 2R-trans; HCl(1:2); H₂O(1:1) |
| 142 | B2 | cb | C═O | cb | 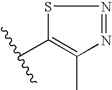 | 2R-trans; succinate (1:2) |
| 143 | B2 | cb | C═O | cb | 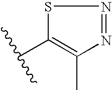 | 2R-trans; malonate (1:2) |
| 144 | B13 | cb | C═O | cb | 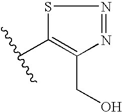 | 2R-trans |
| 120 | B3b | cb | C═O | cb | 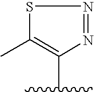 | 2R-trans |
| 79 | B2 | cb | C═O | cb | 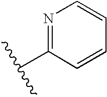 | 2R-trans |
| 166 | B3b | cb | C═O | cb | 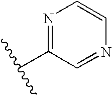 | 2R-trans |
| 80 | B2 | cb | C═O | cb | 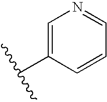 | 2R-trans |
| 81 | B3b | cb | C═O | cb | 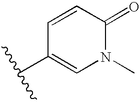 | 2R-trans |
| 82 | B3b | cb | C═O | cb | 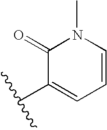 | 2R-trans |
| 83 | B2 | cb | C═O | cb | 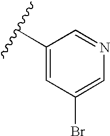 | 2R-trans |
| 14 | B2 | cb | C═O | cb | 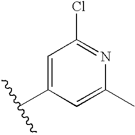 | 2R-trans |
| 84 | B3b | cb | C═O | cb | 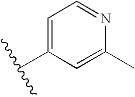 | 2R-trans |
| 85 | B10 | cb | C═O | cb | 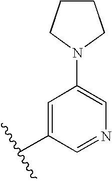 | 2R-trans |
| 86 | B9 | cb | C═O | cb | 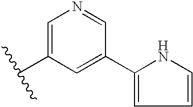 | 2R-trans |
| 87 | B9 | cb | C═O | cb | 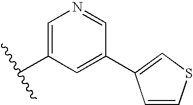 | 2R-trans |
| 88 | B8 | cb | C═O | cb | 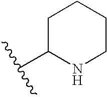 | 2R-trans |
| 89 | B3b | cb | C═O | cb | 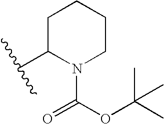 | 2R-trans |
| 90 | B3b | cb | C═O | cb | 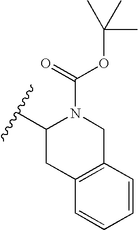 | [2R-[2α,4β(S)]] |
| 91 | B8 | cb | C═O | cb | 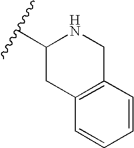 | [2R-[2α,4β(S)]] |
| 92 | B2 | cb | C═O | cb | 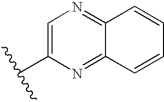 | 2R-trans |
| 93 | B3b | cb | C═O | cb | 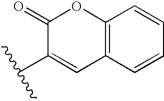 | 2R-trans |
| 94 | B3b | cb | C═O | cb | 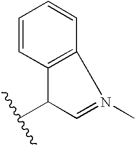 | B-trans |
| 169 | B3b | cb | C═O | cb | 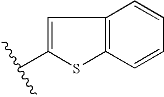 | 2R trans |
| 96 | B2 | cb | C═O | cb | 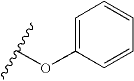 | B-trans |
| 145 | B3b | cb | C═O | cb | 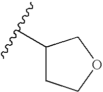 | 2S-trans |
| 146 | B3b | cb | C═O | cb | 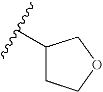 | 2R-cis |
| 147 | B3b | cb | C═O | cb | 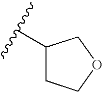 | 2S-cis |
| 173 | | cb | C═O | cb | 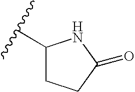 | |
| 97 | B4c | —CH₂— | C═O | cb | 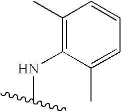 | 2R-trans |
| 98 | B2 | cb | C═O | —CH₂— | —H | 2R-trans |
| 99 | B2 | cb | C═O | 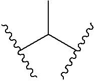 | —H | 2R-trans |
| 159 | B2 | cb | C═O | 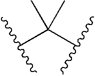 | —H | 2R-trans |
| 167 | B3b | cb | C═O | 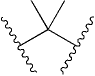 | 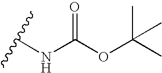 | 2R-trans |
| 160 | B2 | cb | C═O | 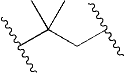 | —H | 2R-trans |
| 100 | B2 | cb | C═O | 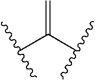 | —H | 2R-trans |
| 101 | B2 | cb | C═O | 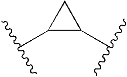 | —H | 2R-trans |
| 148 | B2 | cb | C═O | 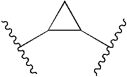 | —H | 25-trans |
| 149 | B2 | cb | C═O | 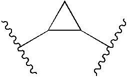 | —H | 2R-cis |
| 150 | B2 | cb | C═O | 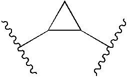 | —H | 25-cis |
| 171 | B3b | cb | C═O | 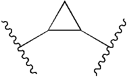 | —H | 2R-trans |
| 172 | B3b | cb | C═O | 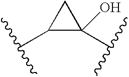 | —H | 2R-trans |
| 102 | B2 | cb | C═O | 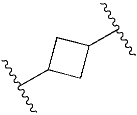 | —H | 2R-trans |
| 151 | B2 | cb | C═O | 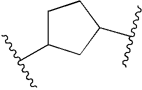 | —H | 2R-trans |
| 103 | B2 | cb | C═O | 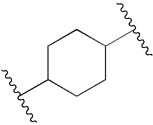 | —H | 2R-trans |
| 104 | B2 | cb | C═O | —CH₂— | 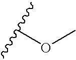 | 2R-trans |
| 105 | B2 | cb | C═O | —CH₂— | 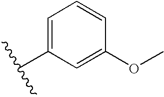 | 2R-trans |
| 106 | B2 | cb | C═O | —CH₂— | 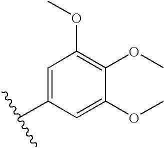 | 2R-trans |
| 107 | B3b | cb | C═O | —CH₂— | 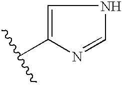 | 2R-trans |
| 13 | B8 | cb | C═O | 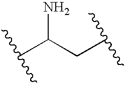 | 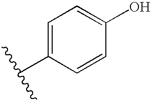 | 2R-trans, HCl(1:3); H₂O(1:1) |
| 108 | B2 | cb | C═O | 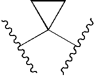 | 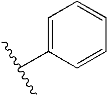 | 2R-trans HCl(1:2) H₂O(1:1) |
| 109 | B2 | cb | C═O | 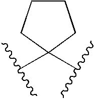 | 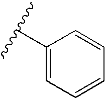 | 2R-trans |
| 110 | B3b | cb | C═O | 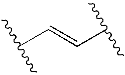 | 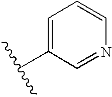 | [2R-[2α,4β(E)[[ |
| 111 | B2 | cb | C═O | 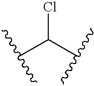 | 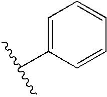 | 2R-trans |
| 112 | B2 | cb | C═O | 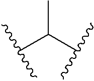 | 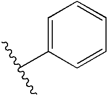 | 2R-trans |
| 152 | B4c | cb | C═O | 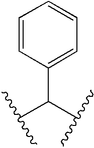 | 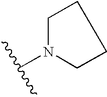 | B-trans |
| 113 | B4c | cb | C═O | 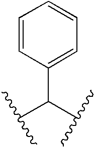 | 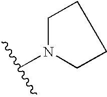 | B-trans HCl(1:3) H₂O(1:3) |
| 114 | B4b | cb | C═O | 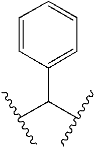 | 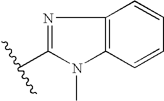 | B-trans |
| 115 | B3b | cb | C═O | 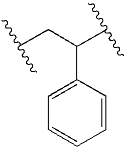 | 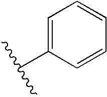 | B-trans |
| 116 | B4c | cb | C═O | 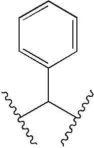 | 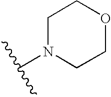 | 2R-trans |
| 6 | B4a | 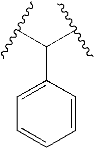 | C═O | cb | 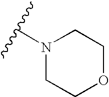 | 2R-trans |
| 117 | B2 | 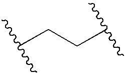 | C═O | cb | 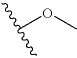 | 2R-trans |
| 168 | B2 | cb | 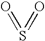 | cb | 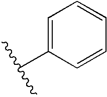 | 2R-trans |
| 118 | B2 | cb | 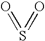 | cb | 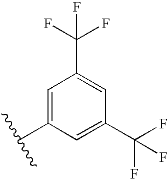 | B-trans |
| 119 | B2 | cb | 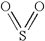 | cb | 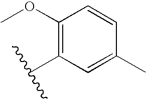 | B-trans |

cb = Covalent Bond

TABLE 2

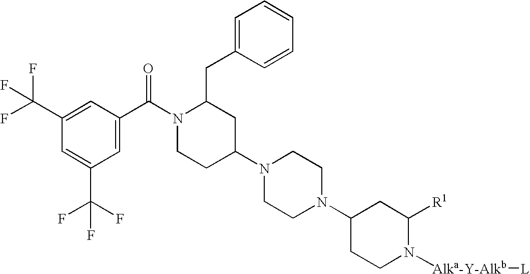

| Co | Exp. | | | | | | |
|---|---|---|---|---|---|---|---|
| No. | No. | R¹ | Alk^{a} | Y | Alk^{b} | L | Physical data |
| 10 | B6 | 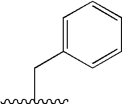 | cb | C═O | cb | 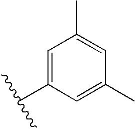 | [2α,4α(2R*,4S*)] |
| 11 | B6 | 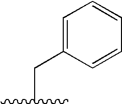 | cb | C═O | cb | 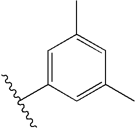 | [2α,4β(2R*,4S*)] |

cb = Covalent Bond

TABLE 3

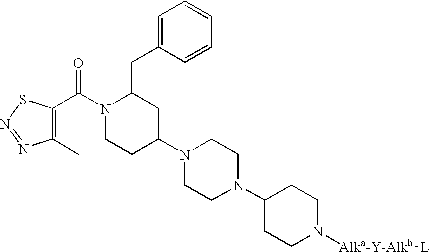

| Co. | Exp. | | | | | |
|---|---|---|---|---|---|---|
| No. | No. | Alk^{a} | Y | Alk^{b} | L | Physical data |
| 153 | B15 | cb | C═O | cb | 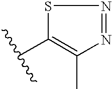 | 2R-trans |

cb = Covalent Bond

TABLE 4

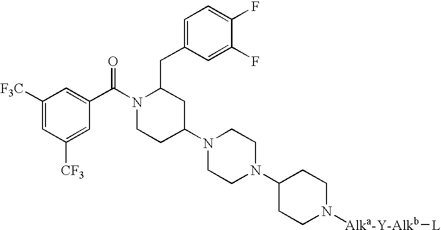

| Co | Exp. | | | | | Physical |
|---|---|---|---|---|---|---|
| No. | No. | Alk^{a} | Y | Alk^{b} | L | data |
| 154 | B1a | —CH₂— | cb | cb | 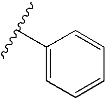 | 2R-cis |
| 155 | B1a | —CH₂— | cb | cb | 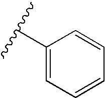 | 2R-trans |
| 156 | B1b | cb | ob | I ob | —H | 2R-trans |
| 157 | B2 | cb | C═O | cb | 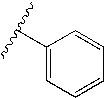 | 2R-trans |
| 158 | B2 | cb | C═O | cb | 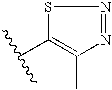 | 2R-trans |

cb = Covalent Bond

TABLE 5

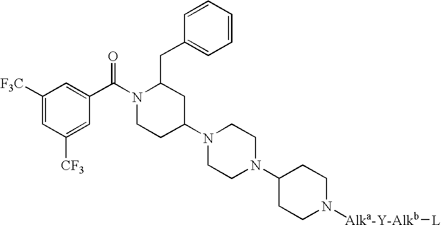

| Co | Exp. | | | | | Physical |
|---|---|---|---|---|---|---|
| No. | No. | Alk^{a} | Y | Alk^{b} | L | data |
| 175 | B1b | cb | cb | cb | H | cis |
| 174 | B1a | —CH₂— | cb | cb | 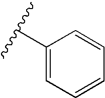 | |
| 176 | B2 | cb | C═O | cb | 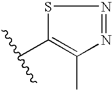 | cis |
| 177 | B2 | cb | C═O | cb | 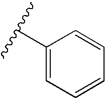 | cis |

cb = Covalent Bond

Analytical Data

For a number of compounds, either melting points, LCMS data or optical rotations were recorded.

Melting Points

If possible, melting points (or ranges) were obtained with a Büchi melting point apparatus B-545. The beating medium is a metal block. The melting of the sample is visually observed by a magnifying lens and a big light contrast. Melting points are measured with a temperature gradient of either 3 or 10 degrees Celsius/minute. Melting points are given in Table 6.

TABLE 6

| Compound | |
|---|---|
| no. | Result (° C.) |
| 1 | 115.9-119.7 |
| 2 | 160.6-163.2 |
| 3 | 149.9-151.7 |
| 4 | 180.5-182.1 |
| 5 | 87.8-121.4 |
| 6 | 87.7-111.2 |
| 7 | 141.0-177.3 |
| 8 | 162.3-164.3 |
| 9 | 122.1-123.8 |
| 10 | 97.0-120.4 |
| 11 | 111.9-125.4 |
| 12 | 66.7-79.0 |
| 13 | 284.5-288.6 |
| 14 | 107.4-116.1 |
| 15 | 188.1-190.3 |
| 19 | 140.3-144.8 |
| 22 | 98.3-119.9 |
| 29 | 142.9-146.5 |
| 31 | 153.1-155.2 |
| 32 | 83.3-95.5 |
| 33 | 82.7-98.6 |
| 34 | 80.7-95.5 |
| 37 | 298.1-319.7 |
| 38 | 83.2-110.2 |
| 39 | 279.4-280.9 |
| 46 | 81.3-107.2 |
| 49 | 145.3-149.6 |
| 50 | 92.1-100.7 |
| 51 | 108.9-127.3 |
| 52 | 93.9-104.6 |
| 53 | 156.6-161.0 |
| 54 | 107.6-122.2 |
| 55 | 96.7-106.3 |
| 56 | 171.3-181.5 |
| 57 | 167.4-169.4 |
| 58 | 92.5-102.6 |
| 59 | 79.1-98.2 |
| 60 | 100.5-121.4 |
| 62 | 91.4-120.3 |
| 63 | 86.0-99.4 |
| 64 | 133.6-159.5 |
| 65 | 102.3-105.8 |
| 69 | 108.6-120.6 |
| 71 | 93.5-127.3 |
| 72 | 91.6-103.2 |
| 73 | 100.5-110.5 |
| 75 | 78.8-93.8 |
| 76 | 76.2-93.8 |
| 77 | 273.6-295.2 |
| 79 | 74.3-100.3 |
| 80 | 106.7-126.1 |
| 81 | 85.3-120.6 |
| 82 | 91.9-121.2 |
| 83 | 86.9-102.1 |
| 84 | 92.2-126.1 |
| 85 | 145.4-147.2 |
| 88 | 70.6-108.7 |
| 89 | 96.1-109.4 |
| 90 | 111.9-120.1 |
| 91 | 91.5-108.1 |
| 92 | 100.7-117.9 |
| 93 | 184.1-192.4 |
| 98 | 177.1-180.6 |
| 99 | 65.9-83.0 |
| 100 | 76.1-100.1 |
| 102 | 72.9-93.5 |
| 103 | 83.7-100.8 |
| 104 | 105.1-108.5 |
| 106 | 77.2-99.1 |
| 108 | 314.8-335.8 |
| 109 | 95.4-107.7 |
| 110 | 84.6-111.8 |
| 111 | 87.3-109.3 |
| 113 | 252.3-291.7 |
| 116 | 102.8-125.6 |
| 117 | 158.2-160.5 |
| 122 | 177.5° C. |

LCMS Conditions

The HPLC gradient was supplied by a Waters Alliance HT 2790 system with a columnheater set at 40° C. Flow from the column was split to a Waters 996 photodiode array (PDA) detector and a Waters-Micromass ZQ mass spectrometer with an electrospray ionization source operated in positive and negative ionization mode. Reversed phase HPLC was carried out on a Xterra MS C18 column (3.5 mm, 4.6×100 mm) with a flow rate of 1.6 ml/min. Three mobile phases (mobile phase A 95% 25 mM ammoniumacetate+5% acetonitrile; mobile phase B: acetonitrile; mobile phase C: methanol) were employed to run a gradient condition from 100% A to 50% B and 50% C in 6.5 min., 100% B in 1 min, 100% B for 1 min. and reequilibrate with 100% A for 1.5 min. An injection volume of 10 mL was used.

Mass spectra were acquired by scanning from 100 to 1000 in 1 s using a dwell time of 0.1 s. The capillary needle voltage was 3 kV and the source temperature was maintained at 140° C. Nitrogen was used a the nebulizer gas. Cone voltage was 10 V for positive ionization mode and 20 V for negative ionization mode. Data acquisition was performed with a Waters-Micromass MassLynx-Openlynx data system. Data is given in Table 7.

TABLE 7

| Compound | |
|---|---|
| no. | LCMS MS(MH+) |
| 16 | 661 |
| 18 | 703 |
| 20 | 711 |
| 21 | 724 |
| 22 | 701 |
| 23 | 703 |
| 24 | 753 |
| 26 | 809 |
| 27 | 699 |
| 28 | 749 |
| 30 | 654 |
| 35 | 703 |
| 36 | 703 |
| 42 | 756 |
| 48 | 719 |
| 61 | 747 |
| 70 | 693 |
| 74 | 692 |
| 94 | 740 |
| 96 | 703 |
| 101 | 651 |
| 105 | 731 |
| 107 | 691 |
| 114 | 803 |
| 115 | 791 |
| 118 | 859 |
| 119 | 767 |
| 124 | 700 |
| 125 | 673 |
| 126 | 673 |
| 127 | 673 |
| 128 | 737 |
| 129 | 709 |
| 130 | 709 |
| 131 | 693 |
| 132 | 687 |
| 133 | 687 |
| 134 | 687 |
| 135 | 701 |
| 136 | 677 |
| 137 | 677 |
| 138 | 677 |
| 139 | 709 |
| 140 | 709 |
| 141 | 709 |
| 142 | 709 |
| 143 | 709 |
| 144 | 725 |
| 145 | 681 |
| 146 | 681 |
| 147 | 681 |
| 148 | 651 |
| 149 | 651 |
| 150 | 651 |
| 151 | 677 |
| 153 | 595 |
| 154 | 709 |
| 155 | 709 |
| 156 | 619 |
| 157 | 723 |
| 158 | 745 |

Optical Rotations

Optical rotations were recorded on a polarimeter (Perkin Elmer) at 20° C. Specifics on concentration, wavelength and solvent are given in Table 8.

TABLE 8

| | | Wavelength | Concentration | |
|---|---|---|---|---|
| Compound no. | [α] | (nm) | (w/v %) | Solvent |
| 18 | −33.77° | 365 | 0.4086 | CH₃OH |
| 159 | −35.56° | 365 | 0.4302 | CH₃OH |
| 160 | −33.66° | 365 | 0.5288 | CH₃OH |
| 161 | −34.75° | 365 | 0.4058 | CH₃OH |
| 162 | −6.72° | 436 | 0.6400 | CH₃OH |
| 163 | −33.2° | 365 | 0.4638 | CH₃OH |
| 164 | −34.1° | 365 | 0.4340 | CH₃OH |
| 165 | −34.43° | 365 | 0.4298 | CH₃OH |
| 166 | −33.95° | 365 | 0.4094 | CH₃OH |
| 167 | −29.91° | 365 | 0.4848 | CH₃OH |
| 168 | −29.12° | 365 | 0.4602 | CH₃OH |
| 169 | −32.32° | 365 | 0.4548 | CH₃OH |
| 170 | −33.3° | 365 | 0.4354 | CH₃OH |
| 171 | −35.06° | 365 | 0.4164 | CH₃OH |
| 172 | −35.84° | 365 | 0.4380 | CH₃OH |
| 173 | −34.53° | 365 | 0.4054 | CH₃OH |

C. Pharmacological Example

### Example C.1

Binding Experiment for h-NK₁, h-NK₂ and h-NK₃ Receptors

The compounds according to the invention were investigated for interaction with various neurotransmitter receptors, ion channels and transporter binding sites using the radioligand binding technique. Membranes from tissue homogenates or from cells, expressing the receptor or transporter of interests, were incubated with a radioactively labelled substance ([³H]- or [¹²⁵I] ligand) to label a particular receptor. Specific receptor binding of the radioligand was distinguished from the non-specific membrane labelling by selectively inhibiting the receptor labelling with an unlabelled drug (the blank), known to compete with the radioligand for binding to the receptor sites. Following incubation, labelled membranes were harvested and rinsed with excessive cold buffer to remove non-bound radioactivity by rapid filtration under suction. Membrane bound radioactivity was counted in a scintillation counter and results were expressed in counts per minute (cpm).

The compounds were dissolved in DMSO and tested at 10 concentrations ranging from 10⁻¹⁰ to 10⁻⁵ M.

The ability of the compounds according to the invention to displace [³H]-Substance P from cloned human h-NK₁ receptors expressed in CHO cells, to displace [³H]-SR-48968 from cloned human h-NK₂ receptors expressed in Sf9 cells, and to displace [³H]-SR-142801 from cloned human h-NK₃ receptors expressed in CHO cells was evaluated.

The plC₅₀ data for the h-NK₁, h-NK₂ and h-NK₃ receptor testing for a representative selection of compounds are presented in Table 9.

All selected compounds show (sub)nanomolar affinity for the h-NK₁ receptor most of them with more than 100-fold selectivity towards the h-NK₂ and h-NK₃ receptors.

### Example C.2

Signal Transduction

This test evaluates in vitro functional NK₁ antagonistic activity. For the measurements of intracellular Ca⁺⁺ concentrations the cells were grown on 96-well (black wall/transparent bottom) plates from Costar for 2 days until they reached confluence. The cells were loaded with 2 MM Fluo3 in DMEM containing 0.1% BSA and 2.5 mM probenecid for 1 h at 37° C. They were washed 3× with a Krebs buffer (140 mM NaCl, 1 mM MgCl₂×6H₂O, 5 mM KCl, 10 mM glucose, 5 mM HEPES; 1.25 mM CaCl₂; pH 7.4) containing 2.5 mM probenecid and 0.1% BSA (Ca⁺⁺-buffer). The cells were preincubated with a concentration range of antagonists for 20 min at RT and Ca⁺⁺-signals after addition of the agonists were measured in a Fluorescence Image Plate Reader (FLTPR from Molecular Devices, Crawley, England). The peak of the Ca⁺⁺-transient was considered as the relevant signal and the mean values of corresponding wells were analysed as described below.

The sigmoidal dose response curves were analysed by computerised curve-fitting, using the GraphPad Program. The EC₅₀ -value of a compound is the effective dose showing 50% of maximal effect. For mean curves the response to the agonist with the highest potency was normalised to 100%. For antagonist responses the IC₅₀-value was calculated using non-linear regression.

TABLE 9

| Co | h-NK₁ | h-NK₂ | h-NK₃ |
|---|---|---|---|
| No. | pIC₅₀ | pIC₅₀ | pIC₅₀ |
| 5 | 10.0 | 6.1 | 6.3 |
| 110 | 10.0 | — | — |
| 97 | 9.5 | 6.3 | 6.4 |
| 45 | 9.5 | — | — |
| 22 | 9.4 | 6.2 | 6.5 |
| 151 | 9.4 | 6.2 | 6.4 |
| 80 | 9.3 | 6.1 | 6.6 |
| 62 | 9.2 | 6.4 | 6.6 |
| 104 | 9.2 | 5.8 | 5.8 |
| 8 | 9.2 | — | — |
| 78 | 9.1 | 6.4 | 6.0 |
| 12 | 9.1 | 6.0 | 6.1 |
| 39 | 9.1 | 6.0 | 6.0 |
| 113 | 9.0 | 6.4 | 6.4 |
| 16 | 9.0 | 6.3 | 6.8 |
| 56 | 9.0 | 6.3 | 6.7 |
| 143 | 9.0 | 6.1 | 6.3 |
| 36 | 9.0 | 6.1 | 6.1 |
| 77 | 9.0 | 6.1 | 5.6 |
| 106 | 9.0 | 6.0 | 6.3 |
| 102 | 9.0 | — | — |
| 6 | 9.0 | — | — |
| 3 | 8.9 | 6.3 | 6.6 |
| 142 | 8.9 | 6.2 | 6.6 |
| 51 | 8.9 | 6.2 | 6.4 |
| 9 | 8.9 | 6.2 | 6.3 |
| 13 | 8.9 | 6.2 | 6.0 |
| 32 | 8.8 | 6.2 | 6.8 |
| 139 | 8.8 | 6.1 | 6.5 |
| 4 | 8.8 | 5.2 | 6.7 |
| 108 | 8.8 | — | — |
| 89 | 8.6 | 6.2 | 6.2 |
| 116 | 8.6 | 6.1 | 6.8 |
| 2 | 8.6 | 5.8 | 5.2 |
| 42 | 8.6 | — | — |
| 140 | 8.5 | 5.4 | 5.3 |
| 85 | 8.5 | — | — |
| 37 | 8.4 | 6.3 | 6.6 |
| 65 | 8.4 | 6.2 | 6.6 |
| 133 | 8.4 | 5.9 | 6.1 |
| 79 | 8.2 | 6.5 | 6.4 |
| 64 | 8.1 | 6.4 | 6.4 |
| 7 | 8.1 | 6.0 | 6.0 |
| 141 | 8.1 | 5.4 | 5.4 |
| 132 | 8.0 | 5.7 | 5.5 |
| 134 | 7.7 | 5.6 | <5 |
| 119 | 7.6 | 6.0 | 6.0 |
| 90 | 7.5 | 6.5 | 6.9 |
| 11 | 7.4 | 6.2 | 6.6 |
| 26 | 7.4 | 6.0 | 6.0 |
| 10 | 7.3 | 6.4 | 6.2 |
| 144 | — | 5.9 | 6.2 |

### Examples C.3

Antiemetic Effects: Loperamide-Induced Retching in Ferrets

Unless otherwise specified, in all subsequent tests Compounds 3 and 77 were evaluated.

The antiemetic effects have been determined using the loperamide-induced retching model (i.e. retching induced by an opioid) in ferrets. To exclude species differences in antiemetic activiey, both compounds have also been tested for antiemetic activity against apomorphine in dogs.

Antagonism of emesis induced by the peripherally selective opioid loperamide (0.31 mg/kg, s.c.) was studied over a 1 h-period starting immediately after the emetic challenge in ferrets pretreated with test compound or solvent. In control animals pre-treated with solvent, loperamide induced pronounced retching (mean±SD: 95±39 counts; n=529) and, to a lesser extent, vomiting (5±4).

Table 10 lists the ED₅₀s (95% CL; mg/kg) of Compounds 3 and 77 obtained for inhibition (<20 retches; 2.0% false positives) and blockade (=0 retches; 0% false positives) of loperamedic-induced retching at several time intervals after oral, s.c. and i.v. administration.

TABLE 10
ED₅₀s (95% CL; mg/kg) for inhibition and blockade of
loperamide-induced retching as a function of time after oral,
s.c. and i.v. administration.

| | |
|---|---|
| Time | ED₅₀s (95% CL; mg/kg) |

| | | |
|---|---|---|
| (h) | Compound 3 | Compound 77 |

Inhibition of retching:
Oral route:

| | | | | |
|---|---|---|---|---|
| 1 | 0.72 | (0.32-1.62) | 0.31 | (0.14-0.71) |
| 2 | 0.96 | (0.52-1.74) | 0.080 | (0.036-0.18) |
| 4 | 1.25 | (0.82-1.92) | 0.26 | (0.17-0.38) |
| 8 | 1.25 | (0.82-1.92) | 0.29 | (0.22-0.40) |
| 16 | 1.26 | (0.82-1.94) | 0.73 | (0.40-1.33) |
| 32 | 3.81 | (2.08-6.97) | ˜2.5 | (— - —)^{a)} |

| | | |
|---|---|---|
| 64 | >10 | not tested |

Subcutaneous route:

| | | | | |
|---|---|---|---|---|
| 1 | 0.55 | (0.30-1.01) | 0.18 | (0.10-0.33) |

Intravenous route:

| | | | | |
|---|---|---|---|---|
| 1 | 0.39 | (0.26-0.28) | 0.15 | (0.10-0.22) |

Blockade of retching:
Oral route:

| | | | | |
|---|---|---|---|---|
| 1 | 1.65 | (0.91-3.02) | 0.72 | (0.40-1.33) |
| 2 | 2.18 | (1.2-4.0) | 0.42 | (0.23-0.76) |
| 4 | 1.25 | (0.82-1.92) | 0.77 | (0.57-1.05) |
| 8 | 2.89 | (1.58-5.29) | 0.34 | (0.25-0.46) |
| 16 | 2.89 | (1.58-5.29) | 1.66 | (0.91-3.04) |

| | | | |
|---|---|---|---|
| 32 | 5.0 | (3.2-7.7) | >2.5 |

| | | |
|---|---|---|
| 64 | >10.0 | not tested |

Subcutaneous route:

| | | | | |
|---|---|---|---|---|
| 1 | 0.96 | (0.52-1.75) | 0.32 | (0.21-0.49) |

Intravenous route:

| | | | | |
|---|---|---|---|---|
| 1 | 0.88 | (0.59-1.3) | 0.26 | (0.17-0.39) |

^{a)}At 2.5 mg/kg, only 1 out of 5 ferrets showed less than 20 retches. However, the number of retches obtained in the 5 ferrets (42, 21, 20, 40, 16) indicates that the ED₅₀ for inhibition of retching (<20 retches) is close to 2.5 mg/kg.

After oral administration, retching was inhibited (<20 retches) by at graphically estimated peak-effect ED₅₀s of 0.16, 1.0 and 0.85 mg/kg, respectively, and completely blocked (=0 retches) at 0.34, 1.4 and 1.5 mg/kg, respectively. At 4 times the peak-effect dose, the compounds showed a rapid onset of action (<1.0 h) and a duration of action of 16 h for Compound 77 and 32 h for Compound 3.

One hour after s.c. injection, retching was inhibited at 0.18, 0.55 and 1.25 mg/kg, respectively, and completely blocked at 0.32, 0.96 and 3.16 mg/kg, respectively. The ration of oral ED₅₀ (at time of peak effect) over subcutaneous ED₅₀ (obtained at 1 h) was small for the three compounds: Compound 77 (1.1) and Compound 3 (1.4-1.8).

Table 11 compares the antiemetic activity of several prior-art NK₁ antagonists. Compound 77 shows an excellent antiemetic activity, comparable with that of GR-203040.

TABLE 11
ED₅₀s (95% CL; mg/kg) for blockade of loperamide
(0.31 mg/kg, s.c.)-induced retching in ferrets at 1 h after
subcutaneous or 2 h after oral administration.

| | ED₅₀ (95% confidence limits; mg/kg) | | | Ratio |
|---|---|---|---|---|
| Compound | s.c. route (−1 h) | | p.o. route (−2 h) | p.o./s.c. |
| Compound 3 | 0.96 | (0.52-1.75) | 2.18 (1.2-4.0) | 2.3 |
| Compound 77 | 0.32 | (0.21-0.49) | 0.42 (0.23-0.76) | 1.3 |
| GR-203040^{a)} | 0.064 | (0.037-0.11) | 0.20 (0.12-0.35) | 3.1 |
| L-760735^{b)} | 0.31 | (— - —)^{g} | not tested | — |
| CP-99994^{c)} | 0.63 | (0.36-1.1) | >10 | >16 |

| | | | |
|---|---|---|---|
| Aprepitant/MK-869^{d)} | >1.25 | 3.1 (1.9-5.0) | <2.5 |
| CP-96345^{e)} | >10 | not tested | — |
| SDZ-NKT-343^{f)} | not tested | >2.5 | — |

^{a)}Ward et al. Discovery of an orally bioavailable NK1 receptor antagonist, (2S,3S)-(2-methoxy-5-tetrazol-1-ylbenzyl)(2-phenylpiperidin-3-yl)amine (GR203040), with potent antiemetic activity. J Med Chem 38: 4985-4992, 1995.
^{b)}McAllister et al. Differential display analysis of the mechanisms of action of antidepressant drugs. Soc Neurosci, Abstracts 25: Part 2 Abs. 733.11, 1999.
^{c)}Piedimonte et al. A new NK₁ receptor antagonist (CP-99,994) prevents the increase in tracheal vascular permeability produced by hypertonic saline. J Pharmacol Exp Ther 266: 270-273, 1993.
^{d)}Kramer et al. Distinct mechanism for antidepressant activity by blockade of central substance P receptors. Science 281: 1640-1645, 1998.
^{e)}Snider et al. Effect of CP-96,345, a nonpeptide substance P receptor antagonist, on salivation in rats. Proc Natl Acad Sci 88: 10042-10044, 1991.
^{f)}Walpole et al. 2-Nitrophenylcarbamoyl-(S)-prolyl-3-(2-naphthyl)alanyl-N-benzyl-N-methylamide (SDZ NKT 343), a potent human NK₁ tachykinin receptor antagonist with good oral analgesic activity in chronic pain models. J Med Chem 41: 3159-3173, 1998.
^{g)}ED₅₀ estimated based on a limited number of animals tested per dose group.

Compound 77 was also found more potent than Compound 3 1 h after i.v. injection, both for inhibition of retching (ED₅₀: 0.15 and 0.39 mg/kg, respectively) and for blockade of retching (ED₅₀: 0.26 and 0.88 mg/kg, respectively).

1. A pharmaceutical composition comprising a pharmaceutically acceptable carrier and, as active ingredients, an opioid analgesic and a therapeutically effective amount of a compound according to Formula (I) 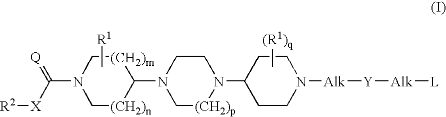
the pharmaceutically acceptable acid or base addition salts thereof, the stereochemically isomeric forms thereof, the N-oxide form thereof and prodrugs thereof, wherein:
n is an integer, equal to 0, 1 or 2;
m is an integer, equal to 1 or 2, provided that if m is 2, then n is 1;
p is an integer equal to 1 or 2;
Q is O or NR³;
X is a covalent bond or a bivalent radical of formula —O—, —S— or —NR³—;
each R³ independently from each other, is hydrogen or alkyl;
each R¹ independently from each other, is selected from the group consisting of Ar¹, Ar¹-alkyl and di(Ar¹)-alkyl;
q is an integer equal to 0 or 1;
R² is selected from the group consisting of alkyl, Ar², Ar²-alkyl, Het¹ or Het¹-alkyl;
Y is a covalent bond or a bivalent radical of formula —C(=O)— or —SO₂—;
each Alk represents, independently from each other, selected from the group consisting a covalent bond; a bivalent straight or branched, saturated or unsaturated hydrocarbon radical having from 1 to 6 carbon atoms and; a cyclic saturated or unsaturated hydrocarbon radical having from 3 to 6 carbon atoms; each radical optionally substituted on one or more carbon atoms with one or more alkyl, phenyl, halo, cyano, hydroxy, formyl and amino radicals;
L is selected from the group consisting of hydrogen, alkyloxy, Ar³-oxy, alkyloxycarbonyl, mono- and di(alkyl)amino, mono-and di(Ar³)amino, Ar³, Ar3-carbonyl, Het² and Het²-carbonyl;
Ar¹ is phenyl, optionally substituted with 1, 2 or 3 substituents each independently from each other selected from the group consisting of halo, alkyl, cyano, aminocarbonyl and alkyloxy;
Ar¹ is naphthalenyl or phenyl, each optionally substituted with 1, 2 or 3 substituents, each independently from each other, selected from the group consisting of halo, nitro, amino, mono- and di(alkyl)amino, cyano, alkyl, hydroxy, alkyloxy, carboxyl, alkyloxycarbonyl, aminocarbonyl and mono-and di(alkyl)aminocarbonyl;
Ar³ is naphthalenyl or phenyl, optionally substituted with 1, 2 or 3 substituents each independently from each other selected from the group consisting of alkyloxy, alkyl, halo, hydroxy, pyridinyl, morpholinyl, pyrrolidinyl, imidazo[1,2-a]pyridinyl, morpholinylcarbonyl, pyrrolidinylcarbonyl, amino and cyano;
Het¹ is a monocyclic heterocyclic radical selected from the group consisting of pyrrolyl, pyrazolyl, imidazolyl, furanyl, thienyl, oxazolyl, isoxazolyl, thiazolyl, isothiazolyl, pyridinyl, pyrimidinyl, pyrazinyl and pyridazinyl; or a bicyclic heterocyclic radical selected from the group consisting of quinolinyl, quinoxalinyl, indolyl, benzimidazolyl, benzoxazolyl, benzisoxazolyl, benzothiazolyl, benzisothiazolyl, benzofuranyl and benzothienyl; each monocyclic and bicyclic heterocyclic radical may optionally be substituted on any atom by a radical selected from the group consisting of halo and alkyl;
Het² is a monocyclic heterocyclic radical selected from the group consisting of pyrrolidinyl, dioxolyl, imidazolidinyl, pyrrazolidinyl, piperidinyl, morpholinyl, dithianyl, thiomorpholinyl, piperazinyl, imidazolidinyl, tetrahydrofuranyl, 2H-pyrrolyl, pyrrolinyl, imidazolinyl, pyrrazolinyl, pyrrolyl, imidazolyl, pyrazolyl, triazolyl, furanyl, thienyl, oxazolyl, isoxazolyl, thiazolyl, thiadiazolyl, isothiazolyl, pyridinyl, pyrimidinyl, pyrazinyl, pyridazinyl and triazinyl; or a bicyclic heterocyclic radical selected from the group consisting of benzopiperidinyl, quinolinyl, quinoxalinyl, indolyl, isoindolyl, chromenyl, benzimidazolyl, imidazo [1,2-a]pyridinyl, benzoxazolyl, benzisoxazolyl, benzothiazolyl, benzisothiazolyl, benzofuranyl and benzothienyl; each monocyclic and bicyclic radical optionally substituted with one or more radicals selected from the group consisting of Ar¹, Ar¹alkyl, halo, hydroxy, alkyl, piperidinyl, pyrrolyl, thienyl, oxo, alkyloxy, alkyloxyalkyl and alkyloxycarbonyl; and
alkyl is a straight or branched saturated hydrocarbon radical having from 1 to 6 carbon atoms or a cyclic saturated hydrocarbon radical having from 3 to 6 carbon atoms; optionally substituted on one or more carbon atoms with one or more radicals selected from the group consisting of phenyl, halo, cyano, oxo, hydroxy, formyl and amino radicals.

2. A pharmaceutical composition according to claim 1, wherein
n is 1;
m is 1;
p is 1;
Q is O;
X is a covalent bond;
each R¹ is Ar¹ or Ar¹-alkyl;
q is 0 or 1;
R¹ is Ar²;
Y is a covalent bond or a bivalent radical of formula —C(=O)— or —SO₂—;
each Alk represents, independently from each other, selected from the group consisting a covalent bond; a bivalent straight or branched, saturated or unsaturated hydrocarbon radical having from 1 to 6 carbon atoms and; a cyclic saturated or unsaturated hydrocarbon radical having from 3 to 6 carbon atoms; each radical optionally substituted on one or more carbon atoms with one or more phenyl, halo, cyano, hydroxy, formyl and amino radicals;
L is selected from the group consisting of hydrogen, alkyloxy, Ar³-oxy, alkyloxycarbonyl, mono- and di(alkyl)amino, mono-and di(Ar³)amino, Ar³ and Het²;
Ar¹ is phenyl, optionally substituted with 1, 2 or 3 alkyl radicals;
Ar² is phenyl, optionally substituted with 1, 2 or 3 alkyl radicals;
Ar³ is phenyl, optionally substituted with 1, 2 or 3 substituents each independently from each other selected from the group consisting of alkyloxy, alkyl, halo, hydroxy, pyridinyl, morpholinyl, pyrrolidinyl, imidazo [1,2-a]pyridinyl, morpholinylcarbonyl, pyrrolidinylcarbonyl, amino and cyano;
Het² is a monocyclic heterocyclic radical selected from the group consisting of pyrrolidinyl, piperidinyl, morpholinyl, pyrrolyl, imidazolyl, pyrazolyl, furanyl, thienyl, isoxazolyl, thiazolyl, thiadiazolyl, pyridinyl, pyrimidinyl, pyrazinyl, and pyridazinyl; or a bicyclic heterocyclic radical selected from the group consisting of benzopiperidinyl, quinolinyl,. quinoxalinyl, indolyl, chromenyl and benzimidazolyl; each monocyclic and bicyclic radical optionally substituted with one or more radicals selected from the group consisting of Ar¹, Ar¹alkyl, halo, hydroxy, alkyl, piperidinyl, pyrrolyl, thienyl, oxo and alkyloxycarbonyl; and
alkyl is a straight hydrocarbon radical having 1 to 6 carbon atoms, optionally substituted with one or more halo radicals.

3. A pharmaceutical composition according to claim 1, wherein R¹ is Ar¹ methyl and attached to the 2-position or R¹ is Ar¹ and attached to the 3-position.

4. A pharmaceutical composition according to claim 1, wherein the R²—X—C(=Q)-moiety is 3,5-di-(trifluoromethyl) phenylcarbonyl.

5. A pharmaceutical composition according to claim 1 wherein, the compound according to Formula (I) is selected from the group of consisting:
{4-[4-(1-Benzoyl-piperidin-4-yl)-piperazin-1-yl]-2-benzyl-piperidin-1-yl}-(3,5-bis-trifluoromethyl-phenyl)-methanone and
(2-Benzyl-4-{4-[1-(4-methyl-[1,2,3]thiadiazole-5-carbonyl)-piperidin-4-yl]-piperazin-1-yl} -piperidin-1-yl)-(3 ,5-bis-trifluoromethyl-phenyl)-methanone.

6. A pharmaceutical composition according to claim 1 wherein, the compound according to Formula (I) is a compound with compound number 5, 110, 97, 45, 22, 151, 80, 62, 104, 8, 78, 12, 39, 113, 16, 56, 143, 36, 77, 106, 102, 6, 3, 142, 51, 9, 13, 32, 139, 4, 108, 89, 116, 2, 42, 140, 85, 37, 65, 133, 79, 64, 7, 141, 132, 134, 119, 90, 11, 26, 10 and 144 as cited in the Experimental section.

7. A pharmaceutical composition according to claim 1, wherein it is formulated for simultaneous, separate or sequential use.

8. A pharmaceutical composition according to claim 1, wherein the opioid analgesic is one or more compounds selected from the group consisting of alfentanil, buprenorphine, butorphanol, carfentanil, codeine, diacetylmorphine, dihydrocodeine, fentanyl, hydrocodone, hydromorphone, levorphanol, lofentanil, meperidine, methadone, morphine, nalbuphine, oxycodone, oxymorphone, pentazocine, propoxyphene, remifentanil and sufentanil; or a pharmaceutical acceptable salt or derivative thereof.

9. A pharmaceutical composition according to claim 8, wherein the opioid analgesic is one or more compounds selected from the group consisting of oxycodone, codeine, morphine, fentanyl, buprenorphine, hydrocodone, hydromorphone and pharmaceutical acceptable salts and derivatives thereof.

10. A pharmaceutical composition according to claim 9, wherein the opioid analgesic is one or more compound selected from the group of morphine sulphate and fentanyl citrate.

11. A pharmaceutical composition according to claim 1, wherein it is in a form suitable to be orally administered.

12. The use of a pharmaceutical composition according to claim 1, for the prevention and/or treatment of pain and/or nociception.

13. The use of a pharmaceutical composition according to claim 1, for the opioid-based prevention and/or treatment of acute and chronic pain, more in particular in inflammatory, post-operative, emergency room (ER), breakthrough, neuropathic and cancer pain treatments.

14. The use of a pharmaceutical composition according to claim 1, for the prevention and/or treatment of emesis in opioid-based treatments of pain.

15. The use of a pharmaceutical composition according to claim 14 for for the prevention and/or treatment of nausea and vomiting in opioid-based treatments of pain.

16. The use of an NK₁-receptor antagonist, in particular an NK₁-receptor antagonist according to Formula (I), the pharmaceutically acceptable acid or base addition salts thereof, the stereochemically isomeric forms thereof, the N-oxide form thereof and prodrugs thereof, for the prevention and/or treatment of respiratory depression in opioid-based treatments of pain.

17. The use of an NK₁-receptor antagonist, in particular an NK₁-receptor antagonist according to Formula (I), the pharmaceutically acceptable acid or base addition salts thereof, the stereochemically isomeric forms thereof, the N-oxide form thereof and prodrugs thereof, for the manufacture of a medicament for reducing and/or overcoming the tolerance observed with opioids in opioid-based treatments of pain.
